(19) United States
(12) Patent Application Publication
(10) Pub. No.: US 2016/0302965 A1
(43) Pub. Date: Oct. 20, 2016

(54) IMPLANTABLE THERAPEUTIC DEVICES

(71) Applicant: ForSight Vision4, Inc., Menlo Park, CA (US)

(72) Inventors: Signe ERICKSON, Menlo Park, CA (US); Randolph E. CAMPBELL, Menlo Park, CA (US); Eugene DE JUAN, JR., Menlo Park, CA (US); Kathleen Cogan FARINAS, Menlo Park, CA (US); Cary J. REICH, Los Gatos, CA (US); Michael BARRETT, Menlo Park, CA (US); Darren DOUD, Menlo Park, CA (US)

(21) Appl. No.: 15/102,191

(22) PCT Filed: Dec. 5, 2014
(86) PCT No.: PCT/US2014/068895

§ 371 Date: Jun. 6, 2016

Related U.S. Application Data
(60) Provisional application No. 61/913,104, filed on Dec. 6, 2013.

(51) Int. Cl. A61F 9/00; A61K 9/08; A61K 31/5575; A61K 9/00

(52) U.S. Cl. CPC A61F 9/0017; A61K 9/0051; A61K 9/08; A61K 31/5575; A61F 9/00781

(57) ABSTRACT

Described are implantable devices (105) and therapeutic agent delivery formulations for the sustained release of therapeutic agents. In one aspect, described is a device to treat an ocular condition of an eye. The device has a proximal region. A tubular body (115) is coupled to the proximal region and has an outer diameter configured to be inserted at least in part into the eye. A reservoir (130) is in fluid communication with the tubular body and has a volume sized to receive an amount of a formulation of a therapeutic agent. One or more outlets (135) are in fluid communication with the reservoir and configured to release therapeutic amounts of the therapeutic agent into the eye for an extended time when the one or more outlets are positioned inside the eye.

### REFERENCE TO PRIORITY DOCUMENT

The present application claims the benefit of priority under 35 U.S.C. §119(e) of U.S. Provisional Application No. 61/913,104, filed Dec. 6, 2013, entitled “Implantable Therapeutic Devices and Methods of Use.” The full disclosure of the provisional application is hereby incorporated by reference in its entirety.

### BACKGROUND

Diseases that affect vision can be treated with a variety of therapeutic agents, but the delivery of drugs to the eye continues to be challenging. The first line standard of care for the treatment of certain ocular diseases, such as glaucoma for example, is the self-administration of daily topical drop medications. This route of administration is very non-invasive, but suffers from the reliance on patients to follow the proper drug treatment regimens. This can result in lack of compliance and the drug being administered less often than the prescribed frequency resulting in sub-optimal treatment benefit, trough intervals between dosing and further symptom progression. Medication delivery even if administered according to proper drug treatment regimen may not provide the ideal pharmacokinetics and pharmacodynamics, for example cause a peak drug concentration several times higher than the desired therapeutic amount.

In addition, opportunities exist to enhance the delivery of certain glaucoma medications by changing the route of administration from topical to intraocular, where the ability to cross the corneal barrier is not a limitation in the formulation of such medications. Further, upon changing the route of administration, there is an opportunity to deliver ocular medications at higher doses that would otherwise prove difficult in a topical formulation due to side effects, such as corneal erythema, burning and stinging.

### SUMMARY

A need remains for minimally-invasive, sustained release delivery of medications to the eye to improve patient outcomes and reduce dependency on patient compliance and adherence, and eliminate “trough” intervals between dosing.

In a first aspect, described herein is a device to treat an ocular condition of an eye. The device has a proximal region; a tubular body coupled to the proximal region having an outer diameter configured to be inserted at least in part into the eye; a reservoir in fluid communication with the tubular body and having a volume sized to receive an amount of a formulation of a therapeutic agent; and one or more outlets in fluid communication with the reservoir and configured to release therapeutic amounts of the therapeutic agent into the eye for an extended time when the one or more outlets are positioned inside the eye.

The formulation of the therapeutic agent can be a free acid formulation. The free acid formulation can be a solution configured to be injected into the reservoir volume after implantation. The free acid formulation can be a free acid formulation of a prostaglandin analogue. The prostaglandin analogue can be travaprost, bimatoprost, tafluprost, or latanoprost. The solution can be dissolved in concentrations higher than a solubility of a prodrug form of the prostaglandin analogue in water at pH 7. The free acid formulation can have a higher solubility in aqueous formulation. The formulation of the therapeutic agent can be a prostaglandin analogue having one or more solubilizing agents. The one or more solubilizing agents can be cyclodextrin, PEG, or ethanol.

The outer diameter of the tubular body can be configured to be inserted in the eye through a small gauge device. The outer diameter of the tubular body can configured to be inserted through an incision or opening in the eye that is no greater than about 0.5 mm. The volume of the reservoir can be less than 5 ul. The extended time can be at least 1 months, 2 months, 3 months, 4, months, 5 months, 6 months, 7 months, 8 months, 9 months, 10 months, 11 months, 12 months or more.

The free acid formulation can be a suspension. The free acid formulation can be a solid biodegradable pellet. The device can further include a boundary layer of fluid between the solid biodegradable pellet and the release control element. The tubular body can be a 5 mm long polyimide tube having a wall thickness of 0.127 mm and an outer diameter of 0.53 mm. The one or more outlets can include a release control element or a porous membrane.

The reservoir can form an interior of the tubular body. The outer diameter of the tubular body can be sized to be delivered using a 25 g needle. The device can have a length of between about 3 mm and about 7 mm. The formulation of the therapeutic agent can be a free acid form of a prostaglandin analogue. The formulation of therapeutic agent can be a solution. The formulation can be a 10% suspension or a solid drug form of the free acid and the volume of the reservoir can be 1 uL and the extended time can be at least 3 months. The reservoir can be less than 5 uL and the extended time can be between 3 to 6 months. The extended time can be at least 3 months the reservoir volume can be less than 5 ul and the therapeutic amount can be a target delivery rate of 40 ng/day to 300 ng/day. The free acid form of the prostaglandin analogue can be bimatoprost free acid at 300 ug/ml in PBS at pH 7. The free acid form of the prostaglandin analogue can be latanoprost free acid is 800 ug/ml in PBS at pH 7. The free acid formulation can be delivered from the reservoir into the vitreous.

The therapeutic amounts can include target delivery rates of 40 ng/day to 300 ng/day and the formulation of the therapeutic agent can be a solution of bimatoprost having a fill concentration less than 15 mg/mL and the extended time can be about 3 months and the reservoir volume can be between about 0.005 mL to about 0.010 mL. A porous structure can be positioned near the one or more outlets and can have a release rate index that is between about 0.0013 mm to about 0.003 mm.

The therapeutic amounts can include a target delivery rate of 40 ng/day to 300 ng/day and the formulation of the therapeutic agent can be a suspension of bimatoprost having a fill concentration less than 20 mg/mL, and the extended time can be about 6 months and the reservoir volume can be between about 0.005 mL to about 0.010 mL. The device can further include a porous structure positioned near the one or more outlets and have a release rate index that is between about 0.003 mm to about 0.024 mm.

The ocular condition treated can include glaucoma, dry or wet age-related macular degeneration (AMD), neuroprotection of retinal ganglion cells, cataract, presbyopia, cancer, angiogenesis, neovascularization, choroidal neovascularization (CNV) lesion, retinal detachment, proliferative retinopathy, proliferative diabetic retinopathy, degenerative disease, vascular disease, occlusion, infection, endophthalmitis, endogenous/systemic infection, post-operative infection, inflammation, posterior uveitis, retinitis, choroiditis, tumor, neoplasm, retinoblastoma, hemophilia, blood disorder, growth disorder, diabetes, leukemia, hepatitis, renal failure, HIV infection, hereditary disease, cerebrosidase deficiency, adenosine deaminase deficiency, hypertension, septic shock, autoimmune disease, multiple sclerosis, Graves' disease, systemic lupus erythematosus, rheumatoid arthritis, shock, wasting disorder, cystic fibrosis, lactose intolerance, Crohn's disease, inflammatory bowel disease, gastrointestinal cancer, degenerative disease, trauma, systemic condition, and anemia.

The one or more outlets can be positioned on the device such that upon implantation they are located in the anterior chamber and the therapeutic agent targets the trabecular meshwork, the ciliary body or both the trabecular meshwork and the ciliary body. The one or more outlets can be positioned on the device such that upon implantation they are located in the vitreous when the device is implanted and wherein the therapeutic agent targets the ciliary body.

The therapeutic agent can increase outflow of aqueous through the trabecular meshwork, reduces aqueous production of the ciliary body, or both. The therapeutic agent can be a prostamide, a prostaglandin analogue, a beta blocker, a carbonic anhydrase inhibitor, or an alpha antagonist.

The one or more outlets can be positioned at a distal end region of the device. The tubular body can have a length so as to avoid the central visual axis when implanted in the eye. The tubular body length can be between about 2 mm and about 10 mm. The proximal region can be configured to remain external to the eye to aid in retention of the device in the eye after implantation. The proximal region can allow access to the reservoir while the device is implanted in the eye. The tubular body can be tapered from a proximal region to a distal region. The tubular body can have a column strength sufficient to permit the device to pierce through eye tissue. The tubular body can extend away from the proximal region along an angle. The proximal region can conform to the curvature of the outer surface of the eye. The reservoir can be an elongate lumen extending along a length of the tubular body. The one or more outlets can include a single exit port at or near a distal end of the tubular body. The reservoir can be located outside the tubular body and within an interior volume of the proximal region The tubular body can have an internal lumen in communication with the reservoir in the proximal region by a proximal opening. The device can include a total volume including the volume of the reservoir and the internal volume of the proximal region. The proximal opening can form an injection port through to the reservoir. The opening can be covered by a penetrable material. The reservoir can be configured to be filled, refilled, flushed or accessed following implantation in the eye. The proximal region can include an access point through which material is injected into and/or removed from the reservoir. The access point can be positioned extraocularly, intra-sclerally, sub-sclerally, or within the anterior chamber and is accessible from an extraocular location. The tubular body can be configured to tunnel through one or more tissues of the eye. The tubular body can be configured to form a scleral tunnel through at least a portion of the sclera before a distal end region of the tubular body enters the vitreous adjacent the ciliary body. The device can include one or more porous structures positioned adjacent the one or more outlets such that the one or more porous structures regulates delivery of the therapeutic agent from the reservoir through the one or more outlets. The therapeutic agent can be released from the reservoir according to a slow diffusion rather than expelled as a fluid stream.

The device can include one or more fixation elements located near a distal end region of the tubular body. The one or more fixation elements can undergo a shape change from a pre-deployment configuration to a post-deployment configuration. The one or more fixation elements can unfurl away from a longitudinal axis of the tubular body. The proximal region can be formed of a flexible material such that the proximal region is deliverable through a small gauge tubular element. The tubular body can have an inner diameter that is less than an outer diameter of the proximal region. The proximal region can be configured to fold or otherwise change shape to a smaller diameter and then return to a retention shape after delivery and release from the tubular body. The reservoir can be configured to remain outside the eye upon implantation of the device. The reservoir can be formed of a material that expands upon filling from a first dimension to a second dimension. The reservoir can be formed of a material that is non-expandable such that walls of the reservoir are fixed. The tubular body can have a portion of its length that extends a distance outside the eye and outside the reservoir. The device can include one or more fixation elements and can be configured to be implanted wholly within the eye. The one or more fixation elements can include clips configured to affix to iris folds upon implantation of the device in the anterior chamber. The clips can be formed of a flexible, resilient material such that they can be formed onto the iris fold.

The device can further include a flexible scaffold configured to fold, bend or otherwise contract to a minimally-invasive size such that the entire device is configured to be delivered into the anterior chamber through a clear corneal incision. The scaffold can include one or more elongate arms coupled to each other and including one or more contact elements. The one or more contact elements can be configured to contact an internal portion of the eye to aid in positioning and retention of the device. The one or more contact elements can be located along the scaffold where the scaffold undergoes a bend or where the arms terminate. The one or more contact elements can make contact with at least three regions within the anterior chamber. The one or more contact elements can be configured to wedge within an angle of the eye near the trabecular meshwork. The arms of the scaffold can vault away from the one or more contact elements such that the scaffold does not contact any region of the eye except where the contact elements are wedged into the angle. The arms of the scaffold can create any of a variety of shapes including triangular, V-shape, U-shape, S-shape, and L-shape. The scaffold can remain outside the optical zone of the eye and avoid the pupil. A length, shape or relative arrangement of the arms can be customizable prior to, during or after implantation. The device can include at least a second reservoir coupled to the scaffold. The tubular body can include the one or more arms and the reservoir can include a lumen extending through an interior of the one or more arms. The one or more outlets can be found on one of the contact elements.

In an interrelated aspect, disclosed is a system capable of providing therapeutic sustained release of one or more of a variety of medications for the treatment or prevention of one or more of a variety of conditions. The one or more of a variety of conditions can include glaucoma. The one or more of a variety of medications can include latanoprost or bimatoprost or a suspension of bimatoprost. The one or more of a variety of medications can include a biodegradable solid pellet of bimatoprost free acid. The one or more of a variety of medications can be a suspension of latanoprost free acid. The one or more of a variety of medications can be delivered to an anterior chamber of an eye or a vitreous of the eye or a combination thereof.

Other features and advantages should be apparent from the following description of various implementations, which illustrate, by way of example, the principles of the claimed subject matter.

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity.

FIGS. 1A-1B are a cross-sectional, schematic views of a portion of the human eye;

FIG. 2A is a cross-sectional, schematic view of an implementation of a device for the delivery of one or more therapeutic agents;

FIG. 2B is a top plan view of the device of FIG. 2A;

FIG. 2C is a cross-sectional, schematic view of the device of FIG. 2A implanted in the eye;

FIG. 3A is a cross-sectional, schematic view of another implementation of a device for the delivery of one or more therapeutic agents;

FIG. 3B is a top plan view of the device of FIG. 3A;

FIG. 3C is a cross-sectional, side view of the device of FIG. 3A implanted for drug delivery into the vitreous;

FIG. 4A is a cross-sectional, side view of another implementation of a device for the delivery of one or more therapeutic agents;

FIG. 4B is a side view of the device of FIG. 4A implanted for drug delivery into the vitreous;

FIG. 5A is a cross-sectional, side view of another implementation of a device for the delivery of one or more therapeutic agents;

FIG. 5B is a cross-sectional, side view of the device of FIG. 5A implanted for delivery of drug into the anterior chamber;

FIG. 6 is a cross-sectional, side view of another implementation of a device for the delivery of one or more therapeutic agents;

FIGS. 7A-7D are cross-sectional, side views of another implementation of a device having internal fixation elements;

FIG. 8 is a cross-sectional, side view of another implementation of a device having internal fixation elements;

FIGS. 9A-9C are cross-sectional, side view of another implementation of a device having a flexible flange element;

FIG. 10A is a cross-sectional, side view of an implementation of a device for the delivery of one or more therapeutic agents to the anterior chamber;

FIGS. 10B-C are schematic views of the device of FIG. 10A implanted in the eye;

FIGS. 11A-11F are schematic views of other implementations of devices for the delivery of one or more therapeutic agents to the anterior chamber;

FIG. 12 is a cross-sectional, side view of an implementation of a device for the delivery of one or more therapeutic agents;

FIGS. 13A-13B are cross-sectional, side views of an implementation of a device for the delivery of one or more therapeutic agents;

FIGS. 14A-14B are cross-sectional, side view of another implementation of a device for the treatment of the eye.

### DETAILED DESCRIPTION

Described herein are implantable devices, systems and methods of use for the delivery of one or more therapeutics for the treatment of diseases. The devices and systems described herein can deliver therapeutics to select regions and structures of the body over a variety of periods of time. Although specific reference is made below to the delivery of treatments to the eye, it also should be appreciated that medical conditions besides ocular conditions can be treated with the devices and systems described herein. For example, the devices and systems can deliver treatments for inflammation, infection, and cancerous growths. It should also be appreciated that any number of drug combinations can be delivered using any of the devices and systems described herein for the treatment of any number of conditions.

The materials, compounds, compositions, articles, and methods described herein may be understood more readily by reference to the following detailed description of specific aspects of the disclosed subject matter and the Examples included therein. Before the present materials, compounds, compositions, articles, devices, and methods are disclosed and described, it is to be understood that the aspects described below are not limited to specific synthetic methods or specific reagents, as such may vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Eye Anatomy

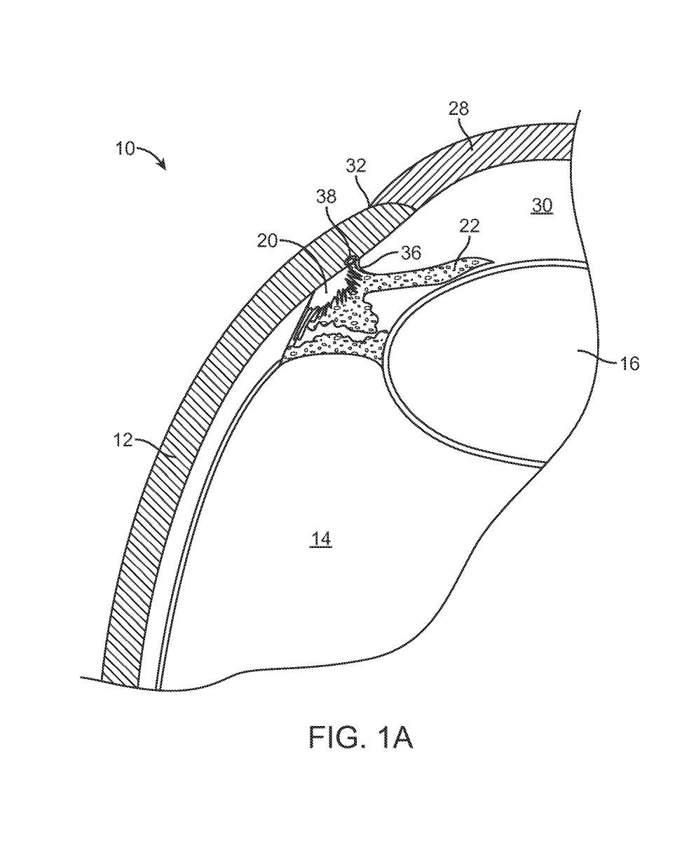

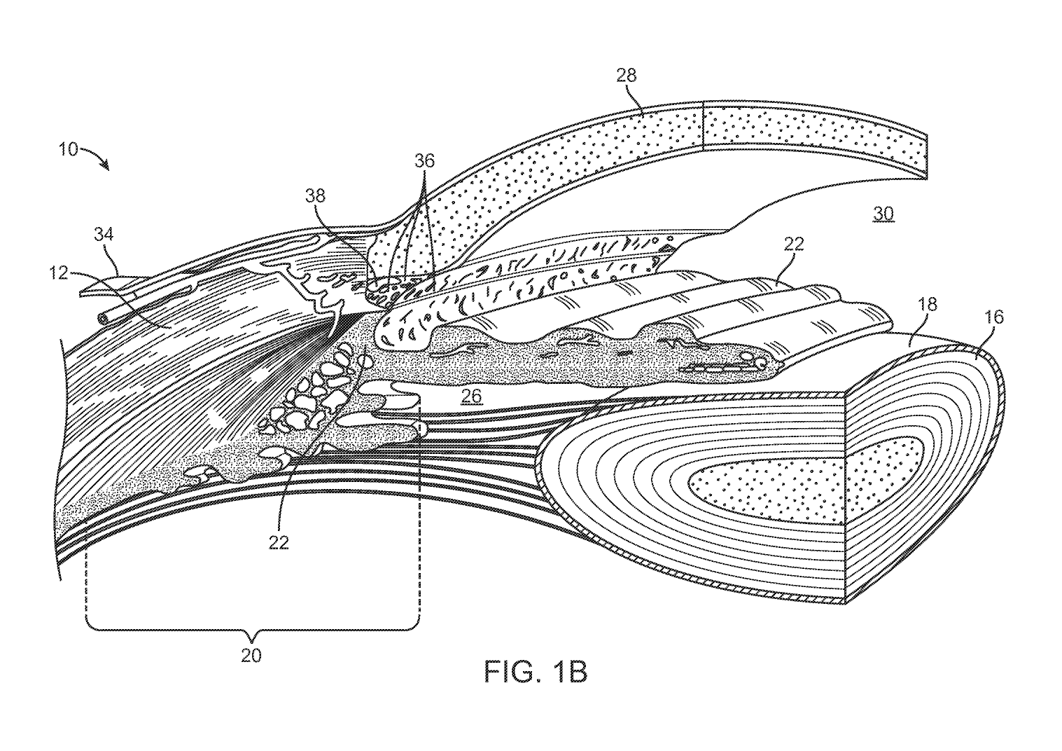

FIGS. 1A-1B are cross-sectional, schematic views of a portion of the human eye showing the anterior and posterior chambers of the eye. The eye 10 is generally spherical and is covered on the outside by the sclera 12. The bulk of the eye 10 is filled and supported by the vitreous body (vitreous humor) 14, a clear, jelly-like substance disposed between the lens 16 and the retina (not shown). The elastic lens 16 is located near the front of the eye 10. The lens 16 provides adjustment of focus and is suspended within a capsular bag 18 from the ciliary body 20, which contains the muscles that change the focal length of the lens 16. A volume in front of the lens 16 is divided into two by the iris 22, which controls the aperture of the lens 16 and the amount of light striking the retina. The pupil 24 is a hole in the center of the iris 22 through which light passes. The volume between the iris 22 and the lens 16 is the posterior chamber 26. The volume between the iris 22 and the cornea 28 is the anterior chamber 30. Both chambers are filled with a clear liquid known as aqueous humor.

The cornea 28 extends to and connects with the sclera 12 at a location called the limbus 32 of the eye. The conjunctiva 34 of the eye is disposed over the sclera 12 and the Tenon's capsule extends between the conjunctiva 34 and the sclera 12. The eye 10 also includes a vascular tissue layer called the choroid (not shown) that is disposed between a portion of the sclera 12 and the retina. The ciliary body 20 is continuous with the base of the iris 22 and is divided anatomically into pars plica and pars plana, a posterior flat area approximately 4 mm long.

The ciliary body 20 continuously forms aqueous humor in the posterior chamber 26 by secretion from the blood vessels. The aqueous humor flows around the lens 16 and iris 22 into the anterior chamber 30 and exits the eye 10 through the trabecular meshwork 36 a sieve-like structure situated at the corner of the iris 22 and the wall of the eye (the corner is known as the iridocorneal angle). Some of the aqueous humor filters through the trabecular meshwork near the iris root into Schlemm's canal 38 that drains aqueous humor into the ocular veins.

Glaucoma is a disease wherein the aqueous humor builds up within the eye. In a healthy eye, the ciliary body 20 secretes aqueous humor, which then passes through the angle between the cornea 28 and the iris 22. Glaucoma appears to be the result of clogging in the trabecular meshwork 36. The clogging can be caused by the exfoliation of cells or other debris. When the aqueous humor does not drain properly from the clogged meshwork, it builds up and causes increased pressure in the eye, particularly on the blood vessels that lead to the optic nerve. The high pressure on the blood vessels can result in death of retinal ganglion cells and eventual blindness. Closed angle (acute) glaucoma can occur in people who were born with a narrow angle between the iris 22 and the cornea 28 (the anterior chamber angle). This is more common in people who are farsighted (they see objects in the distance better than those which are close up). The iris 22 can slip forward and suddenly close off the exit of aqueous humor, and a sudden increase in pressure within the eye follows. Open angle (chronic) glaucoma is by far the most common type of glaucoma. In open angle glaucoma, the iris 22 does not block the drainage angle as it does in acute glaucoma. Instead, the fluid outlet channels within the wall of the eye gradually narrow with time. The disease usually affects both eyes, and over a period of years the consistently elevated pressure slowly damages the optic nerve.

Devices

Devices have been described that are capable of providing therapeutic sustained release of a variety of medications, including for example devices described in PCT Publication No. WO 2012/065006, US Publication No. 2013-0274692, PCT Publication No. WO2013/003620, PCT Publication No. WO 2012/019136, PCT Publication No. WO 2012/019176, and U.S. Pat. No. 8,277,830, each of which is incorporated by reference herein in its entirety.

Described herein are devices for the treatment of various conditions, in particular, glaucoma. The devices described herein are generally low profile and minimally invasive and can provide improved results over, for example, the application of drugs in drop form or other less invasive treatment modalities. Many of the devices described herein can be inserted using an incision or puncture that is minimally invasive. In many implementations, the devices described herein can be inserted using an incision or opening that is 0.5 mm or smaller. However, as will be described in more detail below, the therapeutics to be delivered by the devices described herein are formulated in such a way so as to allow for a sustained delivery of therapeutically effective amounts from a very small reservoir volume over an extended period of time.

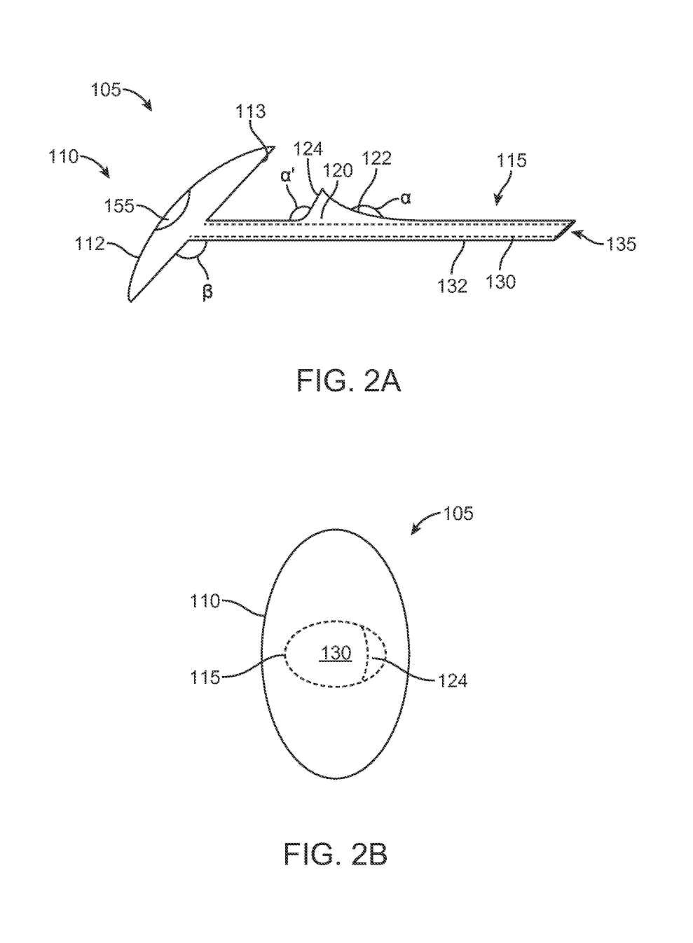

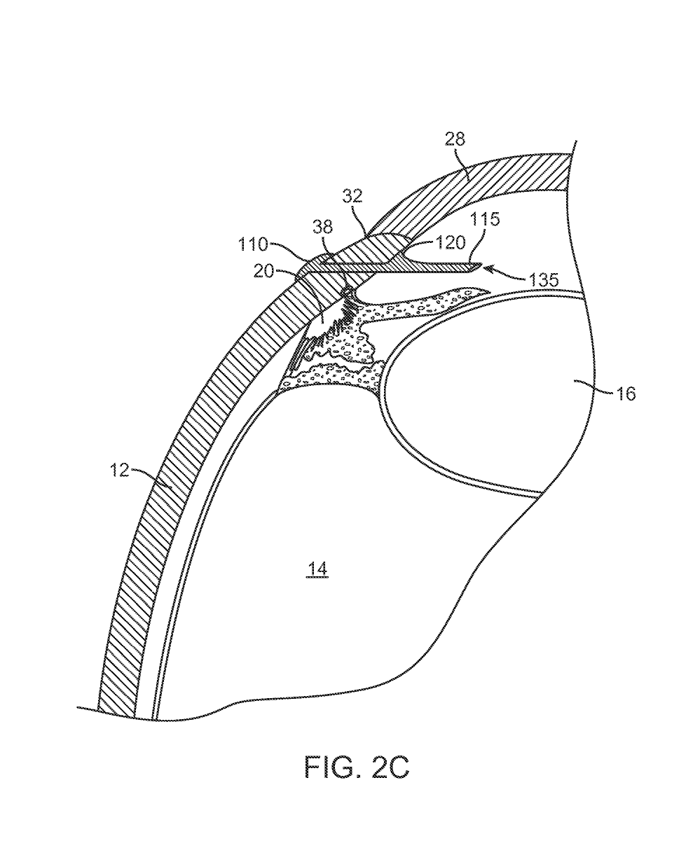

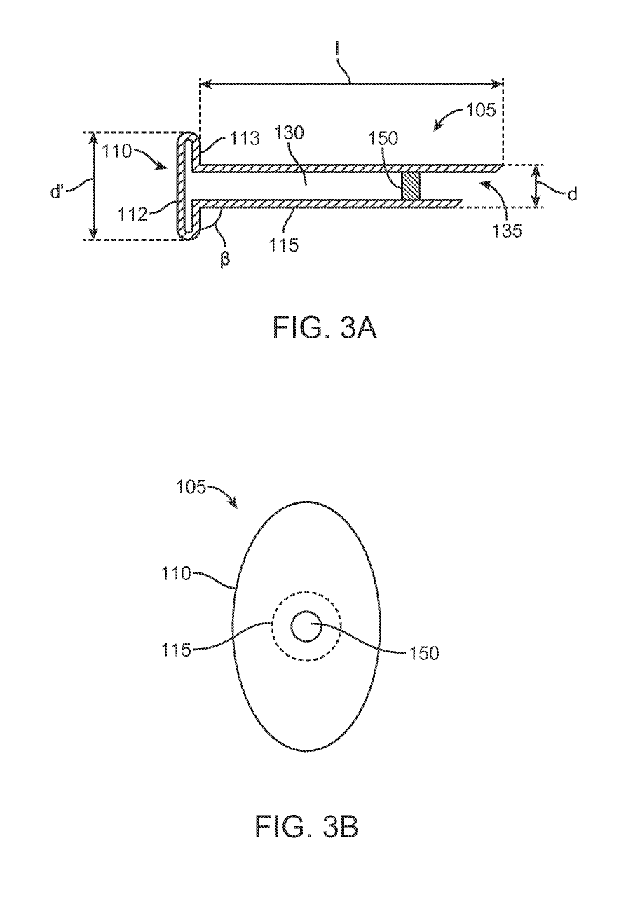

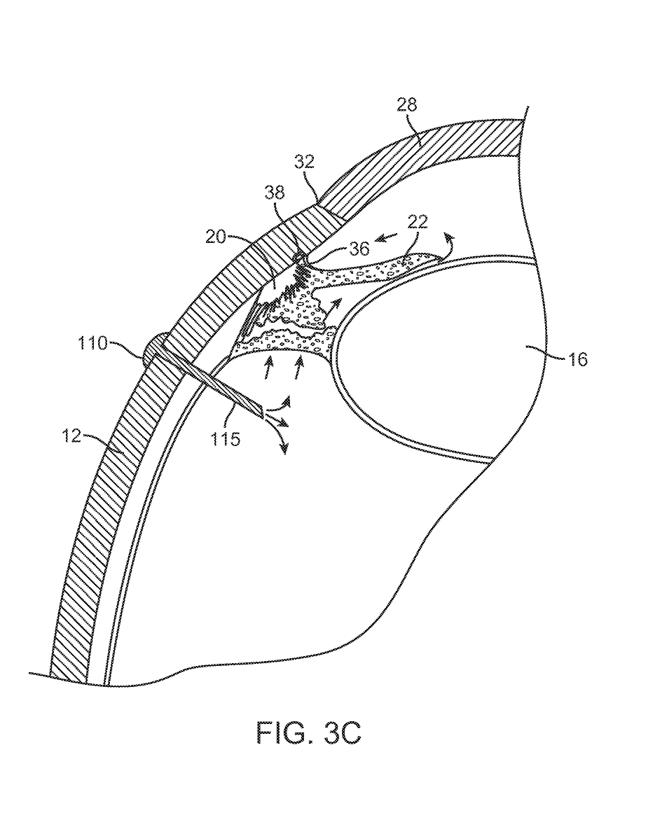

Turning now to the figures, FIGS. 2A-2C illustrate a first implementation of a configured to deliver one or more therapeutic agents to one or more regions of the eye for the treatment of an ocular condition. The device 105 can include a proximal region having a flange element 110 coupled to a tubular body 115 and a reservoir 130 configured to contain one or more therapeutic agents for sustained delivery of the agents to the eye, for example as listed in Table 1 herein below. The device 105 can also include one or more outlets 135 for release of the one or more therapeutic agents into the eye, as will be described in more detail below. The one or more therapeutic agents can be in the form of a liquid, suspension, solid drug core, or other formulation as will be described in more detail below. The device 105 can be inserted such that at least a portion of the body 115, for example the distal end region of the body 115 and/or the one or more outlets 135, are positioned within the eye. The device 105 can be positioned such that the distal end region of the body 115 is located within the anterior chamber 30 near the ciliary body 20 as shown in FIG. 2C. Alternatively, the distal end region of the body 115 can be located within the vitreous as shown in FIG. 3C or another region of the eye as described herein. The device 105 has generally minimally-invasive dimensions such that the device 105 can be inserted at least partially into the eye through a small gauge device as will be described in more detail below.

It should be appreciated that the devices described herein can be implanted in a variety of locations depending upon the drug to be delivered and the treatment desired. For example, intravitreal delivery can be desired for a drug intended to target the ciliary body, whereas another drug may be intended to target the trabecular meshwork and as such a more anteriorly positioned device can be desirable. A more efficient transport and avoidance of potential retinal drug complications (such as with the prostamide class of drugs) can be achieved using a more anterior placement of the device. It also should be appreciated that the various implementations of the devices described herein can be used to treat a variety of target regions and should not be limited to the particular region of the eye. The figures illustrate ways in which the devices described herein can be implanted and are not meant to be limiting. For example, a device shown in the context of trans-scleral implantation for delivery of agents into the anterior chamber (FIG. 2C above) can also be inserted for the delivery of agents to the vitreous (FIG. 3C), the posterior chamber, the supraciliary or suprachoroidal space, Schlemm's canal, the trabecular meshwork, and other locations and vice versa. Similarly, devices shown implanted via trans-scleral delivery can also be inserted through the cornea or another implantation location. Further, structural features described as being incorporated in one implementation of a device can be incorporated into other implementations of devices described herein. It should also be appreciated that the devices described herein can be used to treat a variety of conditions besides glaucoma. The devices described herein can be used in the eye for the treatment of ocular conditions, including but not limited to glaucoma, dry or wet age-related macular degeneration (AMD), neuroprotection of retinal ganglion cells, cataract or presbyopia prevention, cancers, angiogenesis, neovascularization, choroidal neovascularization (CNV) lesions, and a variety of other indications. The devices described herein can be used in other locations in the body for the treatment of other conditions, such as for example in the ear, nasal and paranasal sinuses.

Again with respect to FIGS. 2A-2C, upon implantation the flange element 110 can remain external to the eye to aid in retention of the device in the eye while the body 115 can penetrate eye tissue until at least a portion of the body 115 is positioned intraocularly such that the one or more therapeutic agents can be delivered to the eye. The flange element 110 can form a smooth protrusion for placement along the sclera 12 and under the conjunctiva 34. The flange element 110 can have an outer-facing surface 112 and an inner-facing surface 113. In some implementations when the flange element 110 is positioned against an external surface of the eye, the inner-facing surface 113 of the flange element 110 can contact the sclera and the outer-facing surface 112 of the flange element 110 can be covered by the conjunctiva 34. The conjunctiva 34 covering the outer-facing surface 112 of the flange element 110 can allow access to the device 105 while decreasing the risk of infection to the patient. It should be appreciated that depending on how the device 105 is to be implanted in the eye, the inner-facing surface 113 of the flange element 110 can contact other regions of the eye, for example the cornea 28.

The flange element 110 can have any of a variety of shapes. The flange element 110 can be oval (see FIG. 2B), ovoid, elliptical, circular, or other shape. The flange element 110 can be elongated such that it has a portion that is narrower in one dimension than in another dimension. In some implementations, the flange element can be approximately 1 mm in diameter in a first dimension. In some implementations, the flange element 110 can be generally curved so as to have a contour along a surface of a sphere. The outer-facing surface 112 of the flange element 110 can have a convex shape and the inner-facing surface 113 can have a concave shape such that the flange element 110 can better conform to the curvature of the eye. In other implementations, the flange element 110 can be generally flat. The edges of the flange element 110 can be generally smooth and rounded. The flange element 110 can also be flexible as will be discussed in more detail below.

As mentioned above, the devices described herein can be delivered in a minimally-invasive manner through a small incision or puncture. Generally, the body 115 can have a smaller cross-sectional diameter d compared to the cross-sectional diameter d′ of the proximal flange element 110 (see FIG. 3A). In some implementations, the cross-sectional diameter d of the body 115 can be 0.5 mm across and the cross-sectional diameter d′ of the flange element 110 can be 1.0 mm across. In other implementations, the cross-sectional diameter d of the body 115 can be at least about 0.25 mm across to at least about 0.75 mm across and the cross-sectional diameter d′ of the flange element 110 can be at least about 0.30 mm across to at least about 0.8 mm across. In some implementations, the device 105 can be approximately 25 gauge such that the device 105 can be inserted through a needle bore. A 25 G needle can have an inner diameter of 0.010 mm and 21 G needles can have an ID of 0.020 mm. It should also be appreciated that the respective cross-sectional diameters of the body 115 and the flange element 110 can undergo a shape change as will be described herein in more detail below.

The length l of the body 115 can vary depending on where and how the device 105 is to be implanted in the eye. Generally, the length l is selected so as not to impact or enter the central visual field of the eye upon implantation of the device 105. In some implementations, length l of the body 115 can be between about 2 mm and 10 mm. In other implementations, the length l of the post is about 4 mm. For example, the length l of the body 115 in a device implanted directly through the sclera 12 into the vitreous 14 can be generally shorter than length l of the body 115 in a device to be implanted from a posterior entry site through the sclera 12 into the anterior chamber 30. Further, the cross-sectional shape of the body 115 can vary including circular, oval, rectangular, or other cross-sectional shape. The body 115 can have a substantially uniform diameter along its entire length or the cross-sectional dimension and shape can change along the length of the body 115. In some implementations, the shape of the body 115 can be selected to facilitate easy insertion into the eye. For example, the body 115 can be tapered from the proximal region to the distal region. The device 105 can have column strength sufficient to permit the device 105 to pierce through eye tissue without any structural support. In some implementations, the body 115 can be inserted through the sclera or the cornea without a prior incision or puncture having been made in the eye. The distal tip of the body 115 can be sharpened such that it can penetrate certain eye tissues. Alternatively, the body 115 can be flexible and/or have a blunt or an atraumatic distal tip so as not to puncture certain eye tissues. In such implementations, the device can be wholly contained within a delivery device such that a distal end region of the delivery device provides the column strength and cutting tip sufficient for implantation, as will be described in more detail below.

The body 115 can extend away from the flange element 110 and into the eye along any of a variety of angles. For example, FIG. 2C shows an implementation of a device 105 in which the flange element 110 conforms to the curvature of the outer surface of the eye and the body 115 extends through a region of the sclera 12 such that the distal end region of the body 115 is positioned within the anterior chamber 30. FIG. 3C shows an implementation of a device 105 having a flange element 110 conforming to the curvature of the outer surface of the eye and the body 115 extending through a region of the sclera 12 such that the distal end region of the body 115 is positioned within the vitreous 14. In the implementation of FIG. 2C, an angle β is formed between the inner-facing surface 113 of the flange element 110 and the outer surface of the body 115. In the implementation of FIG. 3C, the body 115 extends generally perpendicular to the inner-facing surface 113 of the flange element 110 forming a substantially right angle β′ between the outer surface of the body 115 and the inner-facing surface 113 of the flange element 110.

As mentioned above, the device 105 can have a reservoir 130 configured to contain one or more therapeutic agents to be delivered to the eye. In some implementations, the reservoir 130 can be the elongate lumen extending along the length of the tubular structure of the body 115 as shown in the implementations of FIGS. 2A-3C. The one or more therapeutic agents can exit the reservoir 130 through one or more outlets 135 in the body 115 that are in fluid communication with the reservoir 130. The number and size of the one or more outlets 135 can vary. For example, the number and size can be selected based on a desired rate of delivery from the reservoir 130. The location of the one or more outlets 135 can vary as well. In some implementations, the one or more outlets 135 can be a single exit port at or near a distal end of the body 115. In other implementations, the one or more outlets 135 can be on a region of the body 115 such as on an outer surface 132 of the body 115 along the length of the body 115 or near the distal end of the body 115. Upon implantation of the device 105 in the eye, the one or more outlets 135 can be positioned so as to align with predetermined anatomical structures. For example, the one or more outlets 135 can be positioned within the anterior chamber, posterior chamber, trabecular meshwork, the iris, the cornea, the ciliary body, the retina, and the vitreous or other regions of the eye. In some implementations, a first outlet 135 can align with a particular structure or structures and a second outlet 135 can align with a different structure or structures.

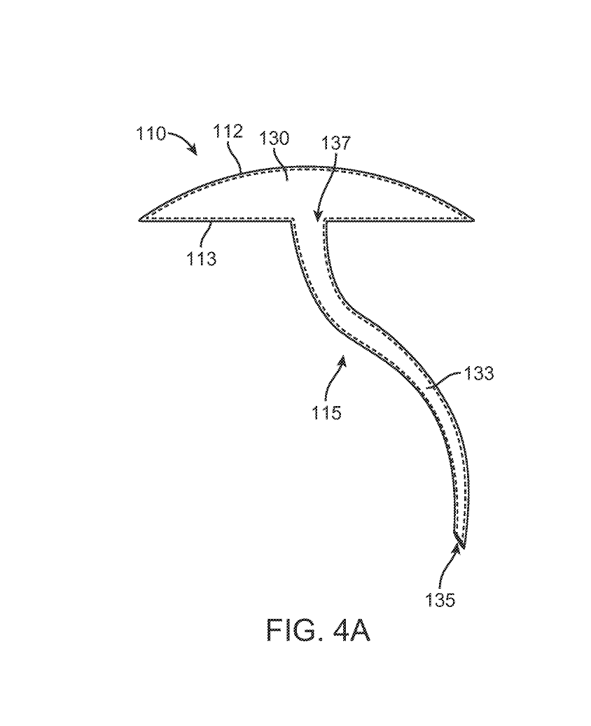

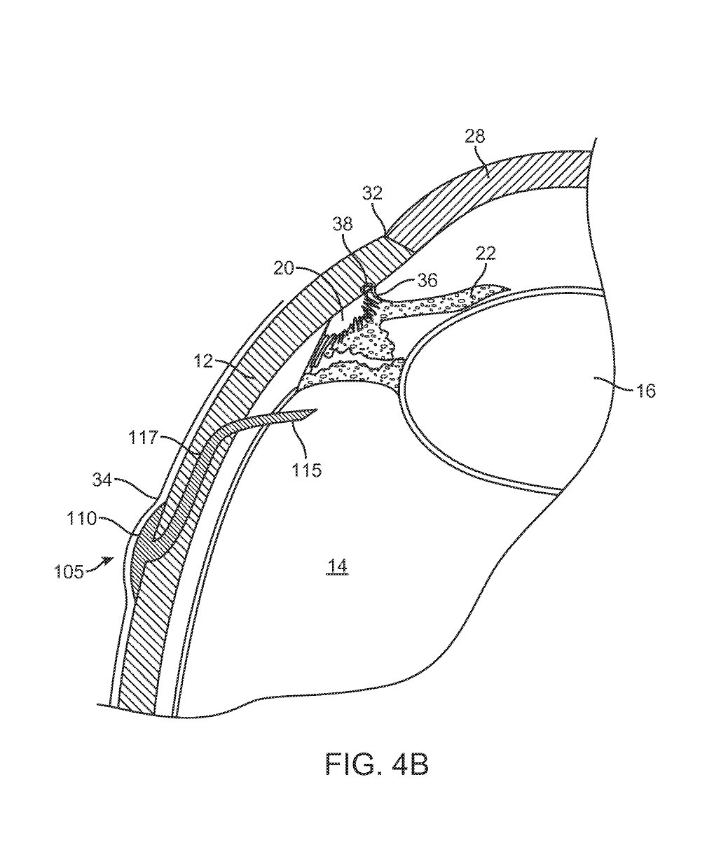

The reservoir 130 need not be located within the tubular structure of the body 115. For example, the reservoir 130 can be located outside the body 115 and within an interior volume of the flange element 110. FIGS. 4A-4B show another implementation of a device 105. The device 105 can include a proximal flange element 110 having an internal volume forming at least a portion of the reservoir 130 containing the one or more therapeutic agents. The device 105 can also include a distal tubular body 115 having an internal lumen 133 in communication with the reservoir 130 such as by a proximal opening 137. As such, the device 105 can have a total reservoir volume that includes both the internal volume of the flange element 110 as well as the volume of the lumen 133. The one or more therapeutic agents contained within the reservoir volume can exit the device 105 through one or more outlets 135 also in communication with the internal lumen 133. The outer-facing surface 112 of the flange element 110 can be implanted under and covered by the conjunctiva and the inner-facing surface 113 of the flange element 110 can abut the scleral surface. The body 115 can be coupled to the flange element 110 on the inner-facing surface 113 of the flange element 110. Upon implantation, the body 115 can penetrate the sclera until at least a distal end portion of the body 115 is inserted within the vitreous (see FIG. 4B), supraciliary or suprachoroidal space, Schlemm's canal, the anterior chamber or another region of the eye as described herein.

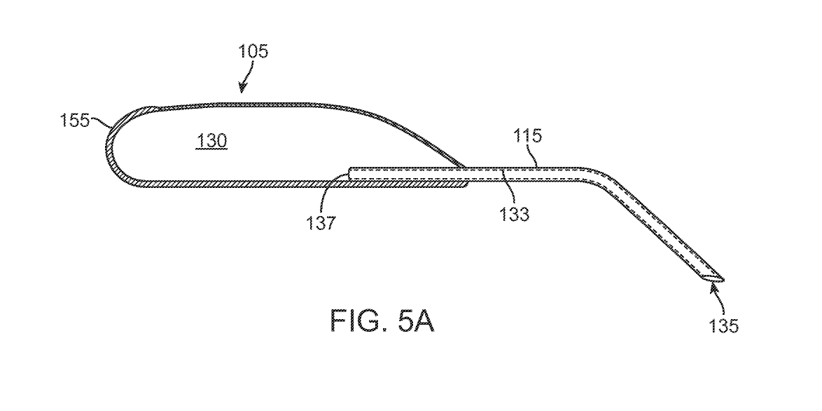

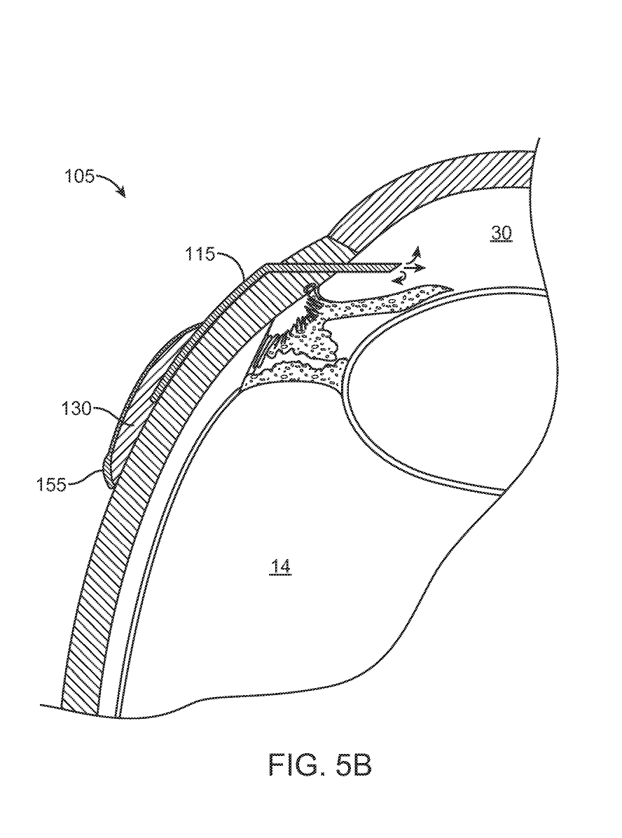

FIGS. 5A and 5B show another implementation of a device 105 in which the reservoir 130 is an extrascleral reservoir and configured to remain outside the eye upon implantation. The reservoir 130 can have an interior volume in fluid communication with the lumen 133 of the tubular body 115. The reservoir 130 can be formed of a material that can expand upon filling of the reservoir 130 from a first dimension to a second dimension. Alternatively, the reservoir 130 can be formed of a material that is non-distensible and/or non-expandable such that the walls of the reservoir 130 are fixed or form a fixed volume. As with other implementations described herein, the tubular body 115 can also include one or more distal openings 135 for delivery of the one or more therapeutic agents from the reservoir 130 to the interior of the eye. The tubular body 115 can also include one or more proximal openings 137 for fluid communication between the interior volume of the reservoir 130 and the inner lumen 133 of the body 115. The proximal end region of the tubular body 115 can couple with the reservoir 130 such as on an anterior-facing region as shown in FIG. 5B or another region of the reservoir 130 such as a posterior-facing region as well as an inferior surface or a superior surface of the reservoir 130. The body 115 can have a length such that it extends a distance outside the reservoir 130 before penetrating a region of the eye. Although FIG. 5B shows the body 115 communicating with the anterior chamber 30 of the eye it should be appreciated that the device 105 can be implanted such that the body 115 inserts through the sclera 12 to communicate with another region of the eye, such as the vitreous, the supraciliary or suprachoroidal space, Schlemm's canal or other region.

The devices described herein can include a drug reservoir configured to be filled, refilled, flushed or otherwise accessed following implantation of the device into the eye. The devices described herein can include an access port for injection and/or removal of material from the reservoir 130. The access point can be positioned extraocularly, intra- or sub-sclerally, or within a region of the eye such as within the anterior chamber as will be described in more detail below and accessed from an extra-ocular location. The access port can be positioned above the sclera as described in U.S. Patent Publication No. 2013/0274692, which is incorporated by reference herein. In some implementations, the flange element 110 can include an injection port 155 (see FIG. 2A) formed by an opening through to the reservoir 130, the opening covered by a penetrable material. Alternatively, one or more regions of the flange element 110 can be formed of the penetrable material. The penetrable material can be configured to be penetrated and resealed such that material does not leak out of the reservoir 130 following penetration of the material. In other implementations in which the reservoir 130 is extrascleral, an outer surface of the reservoir 130 can include an injection port 155 (see FIG. 5A) configured to be accessed with an injection device such as a syringe needle or other type of injection device to access the reservoir 130 while the device 105 is implanted in the eye. The injection port 155 can be formed of or covered by a penetrable material that can be penetrated by the injection device and then seals upon removal of the injection device. The injection port 155 can be located on an outer surface of the device 105. In some implementations, the injection port 155 can be located on an upper surface of the device 105. In other implementations, the injection port 155 can be located on a posterior-facing region of the outer surface away from the body 115 or an anterior-facing region of the outer surface nearer to the body 115.

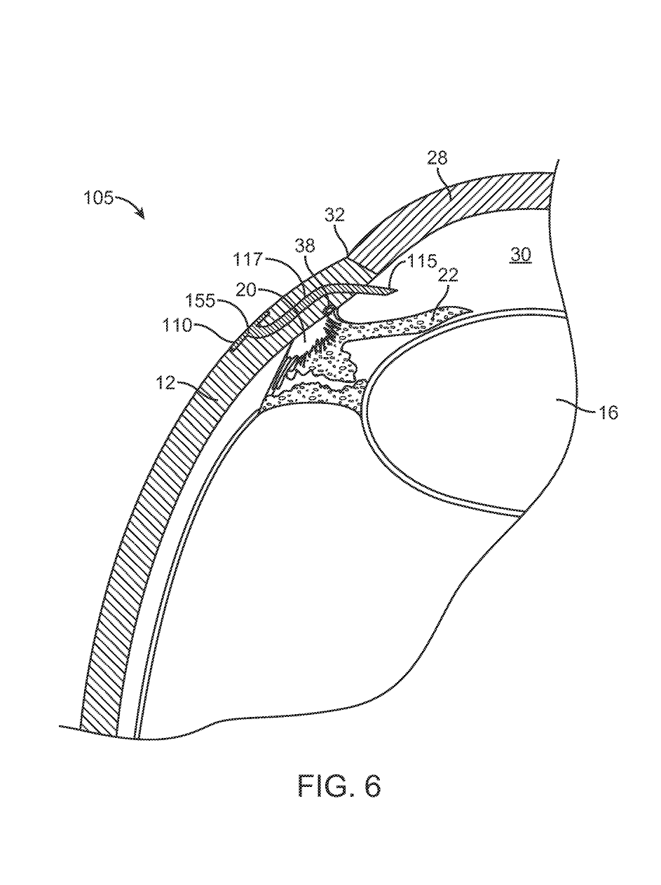

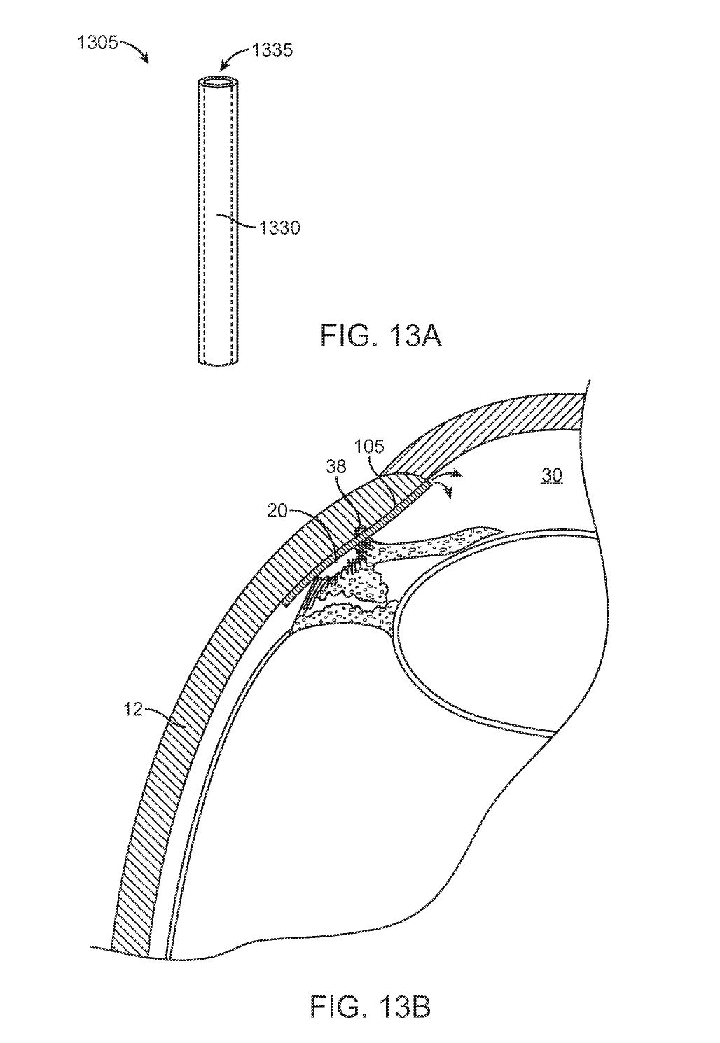

The devices described herein can be implanted such that one or more regions of the device tunnels through one or more tissues of the eye. This allows for the device to be implanted at first location and deliver the one or more therapeutics at a second location distant from the first location. For example again with respect to FIG. 4B, the device 105 can be implanted such that the flange element 110 is positioned near a posterior region of the eye under the conjunctiva 34 and against the sclera 12. The body 115 can form a scleral tunnel 117 through at least a portion of the sclera 12 before the distal end region of the body 115 enters the vitreous 14 adjacent the ciliary body 20. In some implementations, the body 115 can be elongate and of a low profile (e.g. 25 gauge or smaller). FIG. 6 shows another implementation of a device 105 having a body 115 configured to tunnel through eye tissue. The proximal region of the device 105 can reside extrascleral, intrascleral, or subscleral at a generally posterior entry site. The body 115 can form a scleral tunnel 117 from the posterior entry site such that a distal end region of the body 115 can enter the anterior chamber 30. In other implementations, the body 115 of the device can be flexible, curved or otherwise configured to be as less invasive as possible. In some cases, the tunnel may be made by a separate tool that creates the tunnel and/or holds the body of the reservoir coaxially such that the device is placed in the tunnel during the act of forming the tunnel in situ. As with other implementations described herein, the reservoir 130 can be located within the body 115, the flange element 110 or a combination of both the body 115 and the flange element 110. The device 105 can include an access point or injection port 155 for accessing the reservoir 130 of the device 105. Further, the device 105 can be fabricated of light-transmissive material (e.g. fiberoptic material) such that a light shown into the cornea 28 can be transmitted up through the body 115 to illuminate the injection port 155 for improved targeting during extraocular, intrascleral, or subscleral access of the injection port 155. The devices described herein can also include a region of the body 115 configured to enter an anatomical duct such as Schlemm's canal. The devices described herein can also be configured to tunnel between tissues of the eye, such as between the sclera and the choroid or between the sclera and the ciliary body as shown in FIG. 13A-13B to be discussed in more detail below.

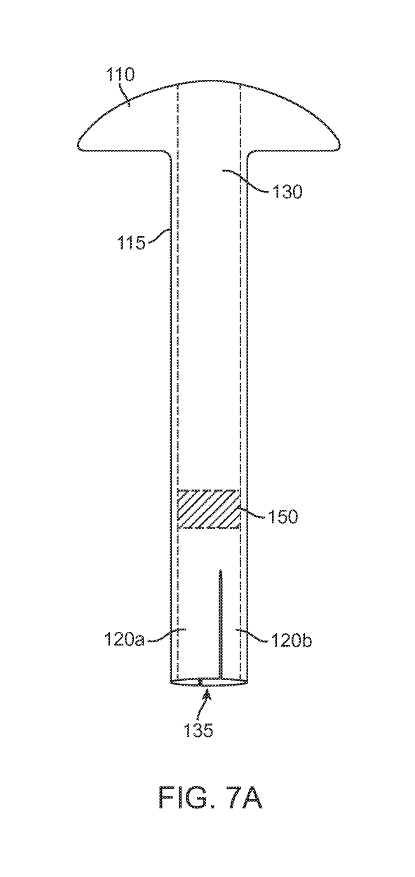

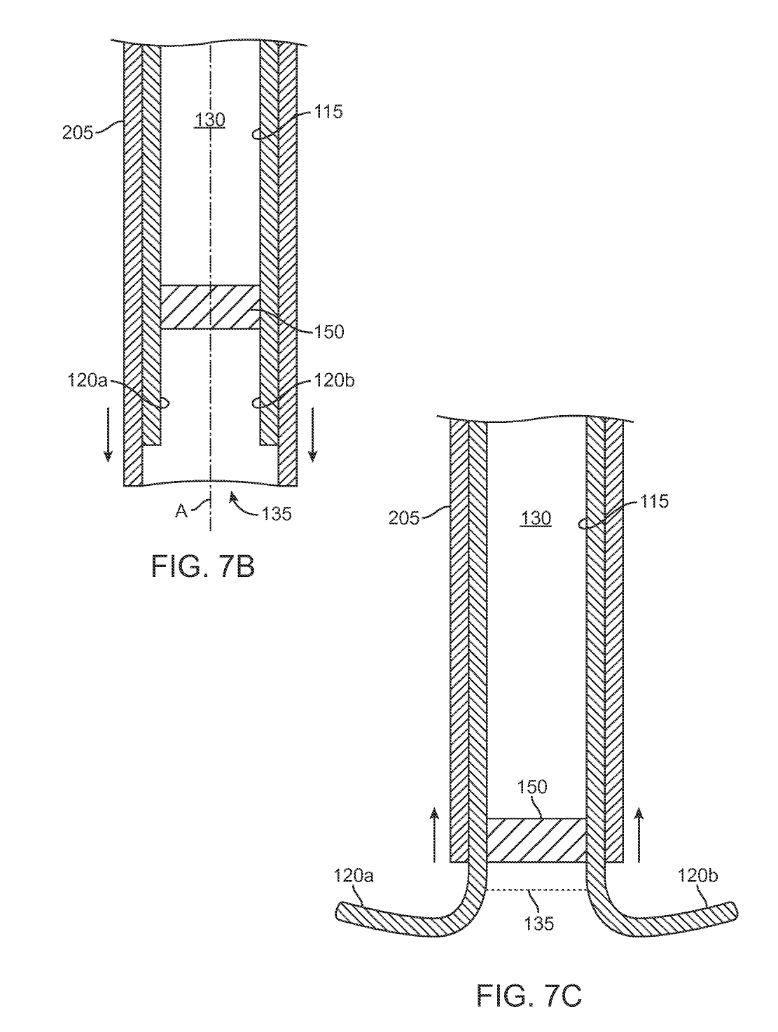

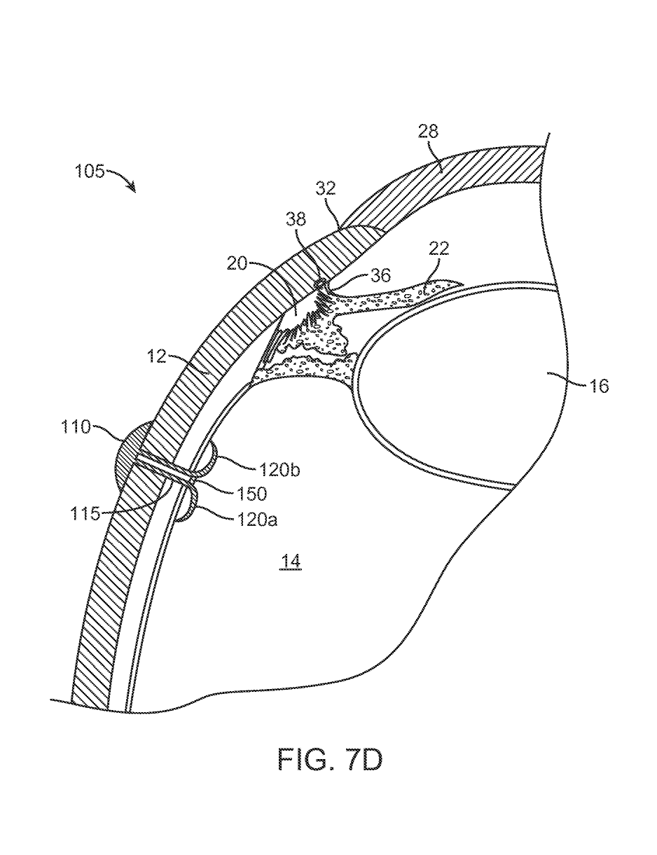

The devices described herein can include one or more porous structure 150. The one or more porous structures 150 can be positioned adjacent the one or more outlets 135 such that the one or more porous structures 150 can control or regulate the delivery of the one or more therapeutic agents from the reservoir 130 through the one or more outlets 135. The contents of the reservoir 130 can be delivered according to a slow diffusion rather than expelled as a fluid stream. In some implementations, the one or more porous structures 150 can be disposed within the reservoir 130, such as within the reservoir 130 of the body 115 as shown in the implementations of FIGS. 3A-3C or FIGS. 7A-7D. The one or more porous structures 150 can be positioned near or at the distal end of the body 115 or somewhere along the length of the reservoir 130. In the implementations where the reservoir 130 is formed by an internal volume of the flange element 110, the porous structure 150 can be positioned near a proximal opening 137 into the lumen 133 of the body 115 or within the reservoir 130 in the flange element 110. In some implementations, the porous structure 150 can be a covering or lining having a particular porosity to the substance to be delivered and can be used to provide a particular rate of release of the substance.

The porous structure 150 can be a release control mechanism, including but not limited to a wicking material, permeable silicone, packed bed, small porous structure or a porous frit, multiple porous coatings, nanocoatings, rate-limiting membranes, matrix material, a sintered porous frit, a permeable membrane, a semi-permeable membrane, a capillary tube or a tortuous channel, nano-structures, nano-channels, sintered nanoparticles and the like. The porous structure 150 can have a porosity, a cross-sectional area, and a thickness to release the one or more therapeutic agents for an extended time from the reservoir. The porous material of the porous structure 150 can have a porosity corresponding to a fraction of void space formed by channels extending through the material. The void space formed can be between about 3% to about 70%, between about 5% to about 10%, between about 10% to about 25%, or between about 15% to about 20%, or any other fraction of void space. The porous structure 150 can be selected from any of the release control mechanisms described in more detail in U.S. Pat. No. 8,277,830, which is incorporated by reference herein.

As mentioned above, the flange element 110 can be configured to aid fixation of the device 105 in an implanted location. The device 105 can also include one or more fixation elements 120 to aid fixation of the device 105 in the implanted location. In some implementations, the device can be used in conjunction with one or more fixation elements coupled to the device, such as a suture or other element to further stabilize and prevent the device from moving after it is implanted in a desired location. In other implementations, the one or more fixation elements 120 can be integral with the device or coupled to a region of the body 115 such as shown in FIG. 2A-2C. Upon insertion of the body 115 through the eye tissue, the flange element 110 can remain outside the sclera and the fixation element 120 can be positioned inside one or more regions of the eye, such as the inner surface of the sclera, cornea, ciliary body, or choroid etc. such that the flange element 110 and the one or more fixation elements 120 work in coordination to provide a snug fit and prevent the device 105 from being extruded from the eye. The fixation element 120 can be a barb coupled to a region of the body 115 and having a first sloped or angled surface 122 on a distal end region that allows for insertion of the body 115 into the eye in a first direction (see FIG. 2A). The fixation element 120 can have a second angled surface 124 on a proximal end region that prevents the body 115 from withdrawing in the opposite direction and out of the eye. In an implementation, the first angled surface 122 forms an angle relative to the outer surface 132 of the body 115 that allows for relatively easy insertion of the device through the sclera in a first direction, whereas the second angled surface 124 forms an angle with the outer surface 132 of the body 115 that prevents or impairs withdrawal of the device 105 through the sclera in a second, opposite direction. It should be appreciated that any of the devices described herein can incorporate one or more fixation elements 120 along one or more regions of the body 115 to aid in the retention of the device in the implanted location.

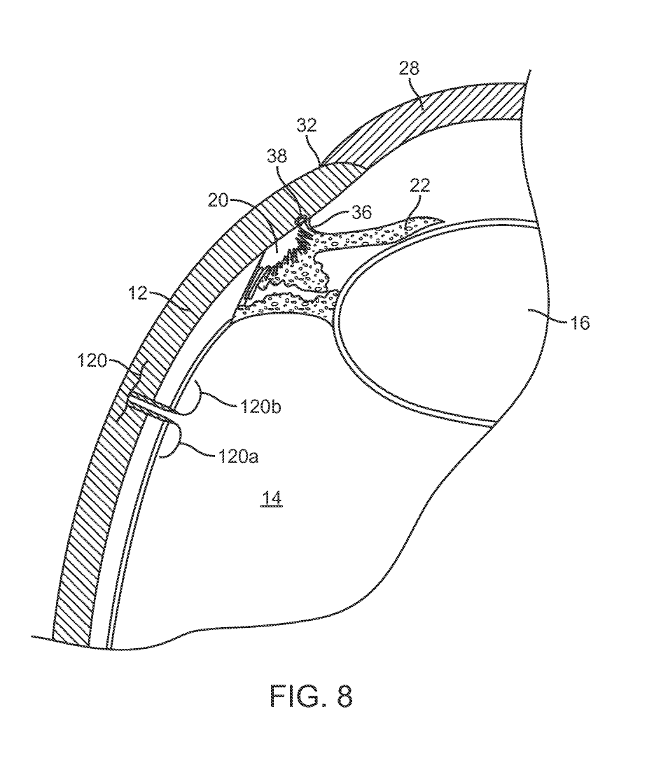

The device 105 can have one or more fixation elements 120 located near a distal end region of the body 115 that can undergo a shape change from a pre-deployment configuration to a post-deployment configuration. In some implementations, the one or more fixation elements 120 can be coupled to the distal end of the body 115. Alternatively, the distal end region of the body 115 can be split such that two or more tabs 120a, 120b are created as shown in FIGS. 7A-7D. The tabs 120a, 120b can be configured to be pressed towards one another (or towards a longitudinal axis A of the body 115) and held in place during delivery such as by a tubular element 205 of the delivery device. After insertion of the body 115 through eye tissue, such as the sclera, and upon ejection from (or withdrawal in a proximal direction of) the tubular element 205 the distal end region of the body 115 is exposed. The tabs 120a, 120b can undergo a shape change such as unfurl away from the longitudinal axis A of the body 115. The tabs 120a, 120b can also bend, curl or otherwise extend away from the longitudinal axis A of the body 115 such as shown in FIG. 7C and FIG. 7D providing fixation of the device 105 within the eye. Similarly, one or more fixation elements 120 can be positioned near a proximal region of the device. FIG. 8 shows a fixation element 120 that is a thin barb configured to penetrate the eye tissue near a proximal end of the device 105. In some implementations, the fixation element can be positioned subsclerally or within a region of the sclera 12. It should be appreciated that any combination of the one or more fixation elements 120 can be incorporated into any of the devices described herein. For example, the device shown in FIG. 8 can have a proximal fixation element 120 such as a scleral barb penetrating a region of the sclera as well as a distal fixation element 120 such as the shape changing tabs 120a, 120b described above. The fixation elements can be fabricated at least in part of a flexible or resilient material, for example a shape-memory material, thermoplastic, flexible metal or plastic material configured to undergo a change in shape upon deployment. In some implementations, the fixation elements can be formed of Nitinol or another temperature sensitive shape memory alloy. The change in shape can be due to shape memory, thermal, magnetic or electromagnetic activation such as from a martensitic to an austenitic state. It should be appreciated that any of a variety of shape memory materials are considered herein.

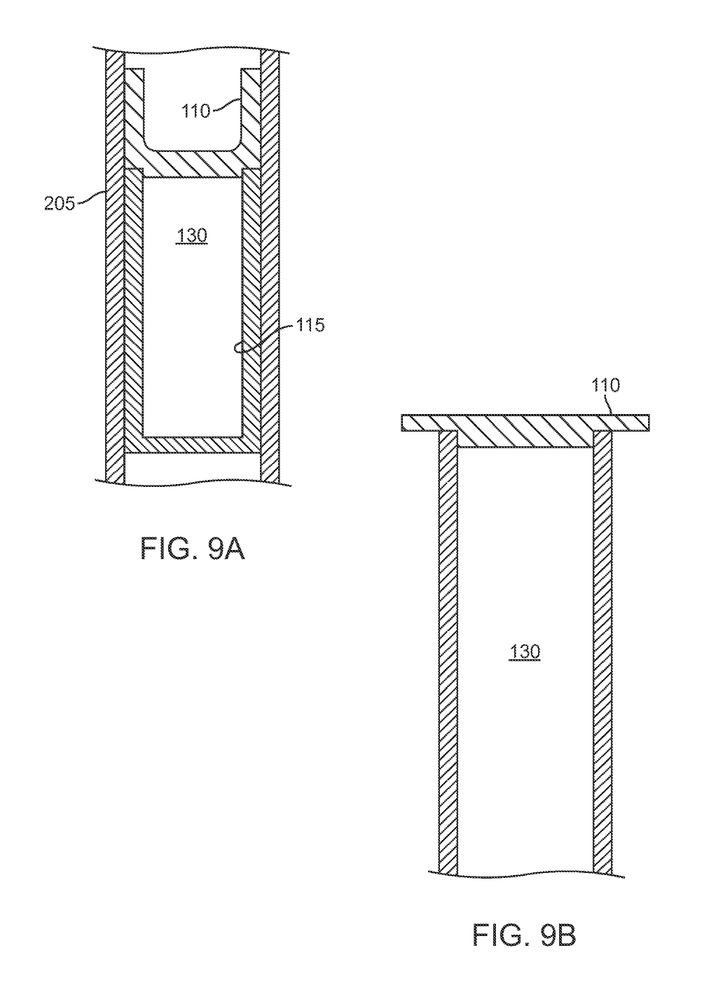

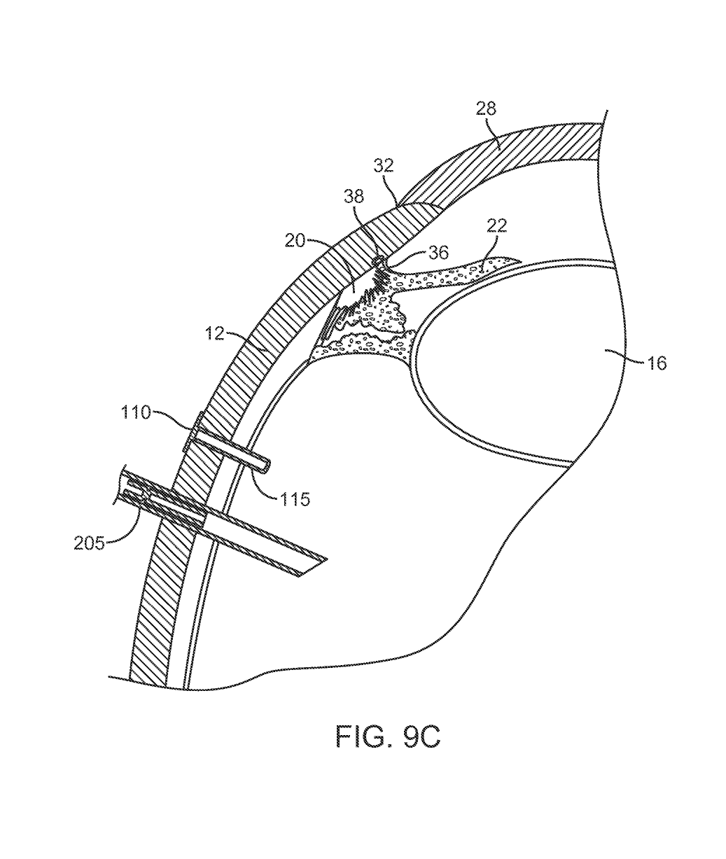

As described above, the one or more fixation elements 120 can undergo shape change from a pre-deployment configuration during which the cross sectional diameter of the device is minimized to a post-deployment configuration after which the cross-sectional diameter of the device is increased to improve retention of the device in the eye. In some implementations, the proximal flange element 110 can undergo a shape change from a pre-deployment configuration suitable for minimally invasive delivery of the device to a post-deployment configuration suitable for retention. As best shown in FIGS. 9A-9C, the flange element 110 can be formed of a flexible material such that the flange element 110 can be delivered through a tubular element 205 having an inner diameter that is less than the outer diameter of the flange element 110. The flange element 110 can fold or otherwise change shape to a smaller diameter and then return to a retention shape after delivery and release from the tubular element 205. The flange element 110 can be fabricated of a flexible, resilient or elastic material having memory as described above. In some implementations, the flange element 110 is formed of a material such as silicone or other material that can undergo a temporary deformation before returning to its original shape.

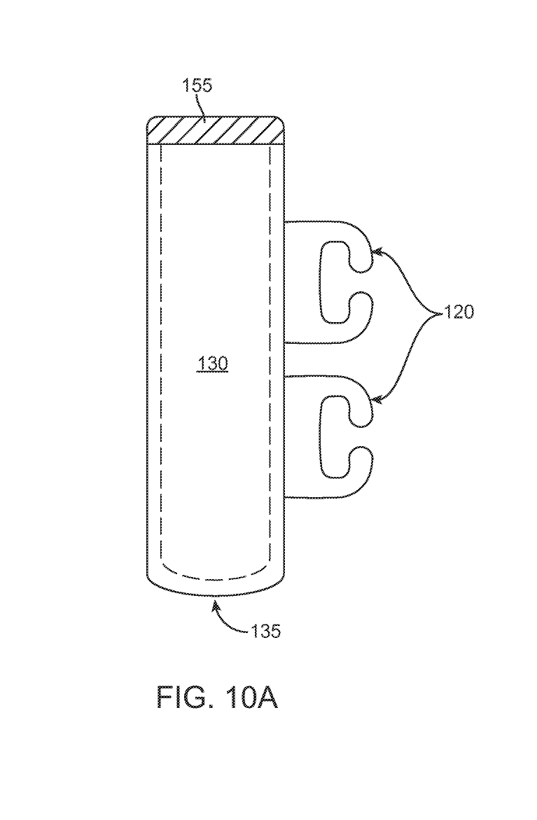

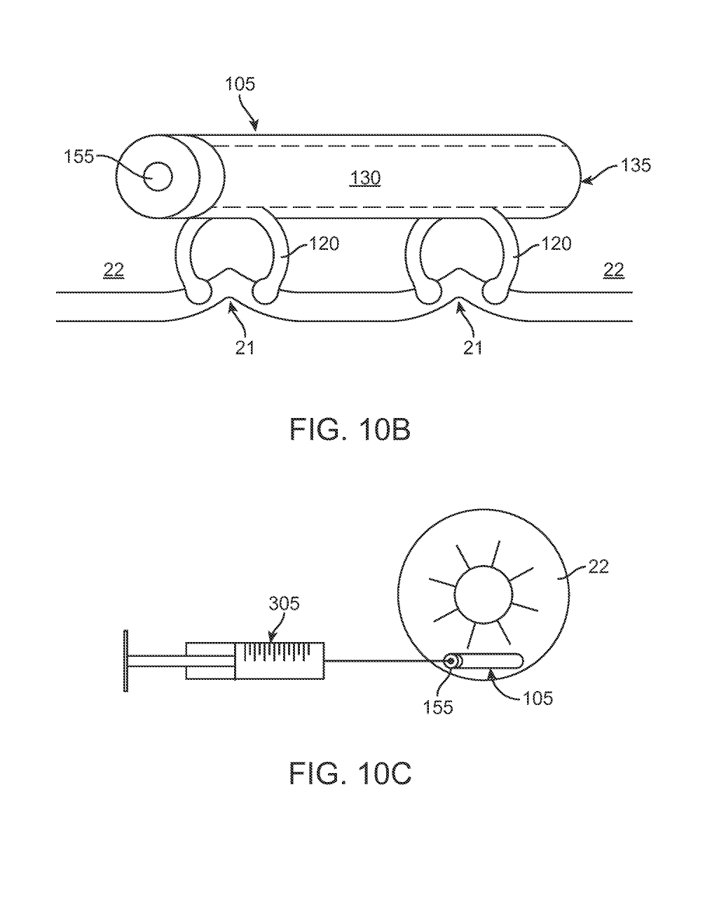

FIGS. 10A-10C show another implementation of a device 105 incorporating one or more fixation elements 120 on an outer region of the device 105. The device 105 is configured to be implanted wholly within the eye, such as within the anterior chamber. The one or more fixation elements 120 can be iris clips configured to affix to iris folds 21. For example, the device 105 can be implanted within the anterior chamber 30 and the one or more fixation elements 120 affixed to the iris folds 21 (see FIG. 10B-10C). The iris clips can be formed of a flexible, resilient material or malleable such that they can be formed onto the iris fold. A region of the device 105 can include an injection port 155 such that the reservoir 130 can be accessed prior to, during or following implantation in the eye, such as for filling, refilling, flushing or removing material from the reservoir 130. The injection port 155 can include a septum fabricated of a penetrable material configured to be passed through by an injection device 305 such as a syringe needle or other element and re-seals following removal of the injection device 305. The injection port 155 can be positioned relative to the anatomy of the eye such that the injection device 305 can access and penetrate the port 155 following implantation in the eye.

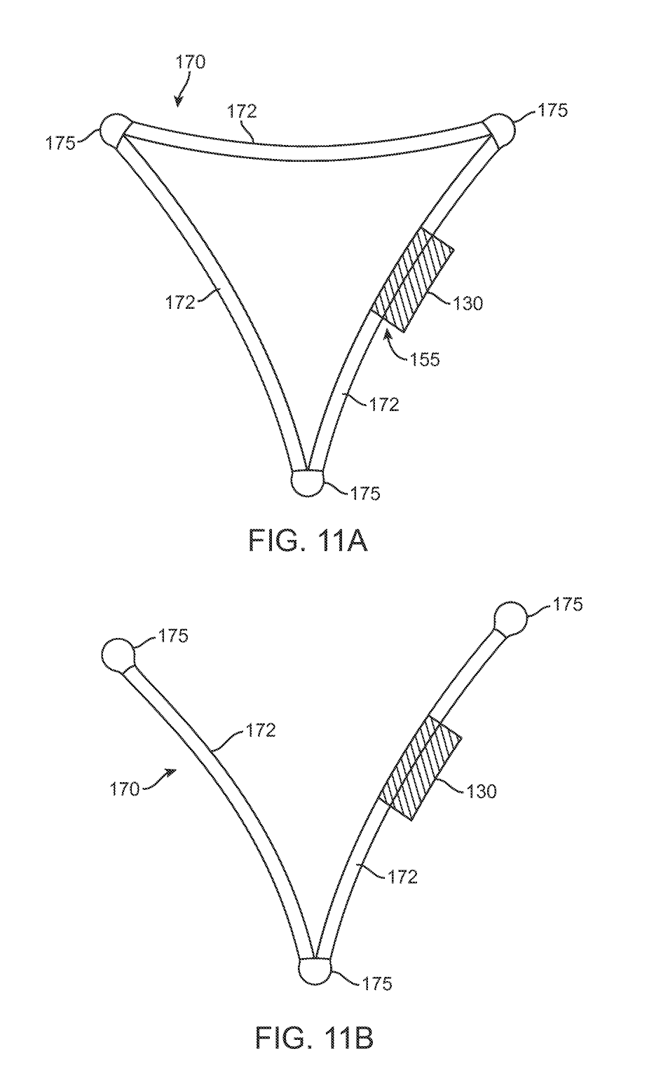

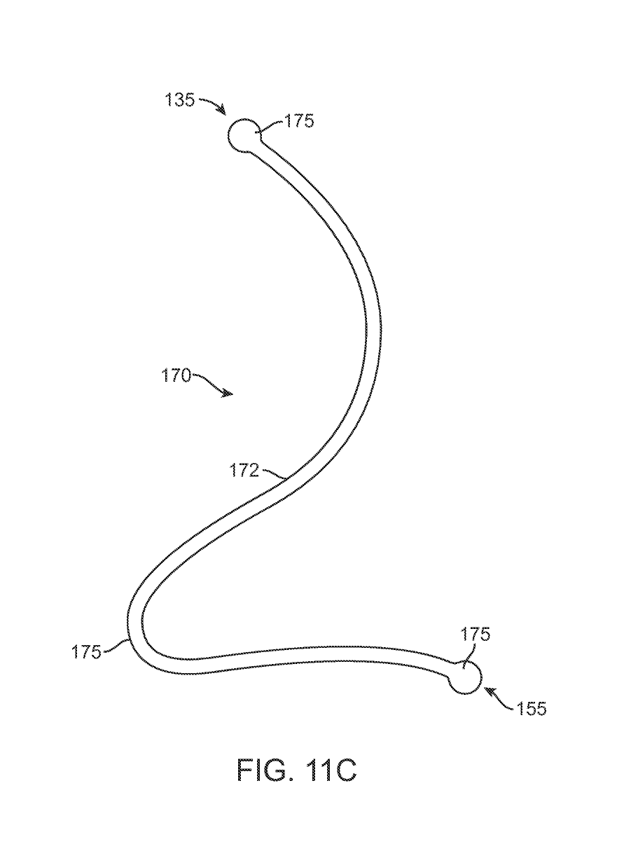

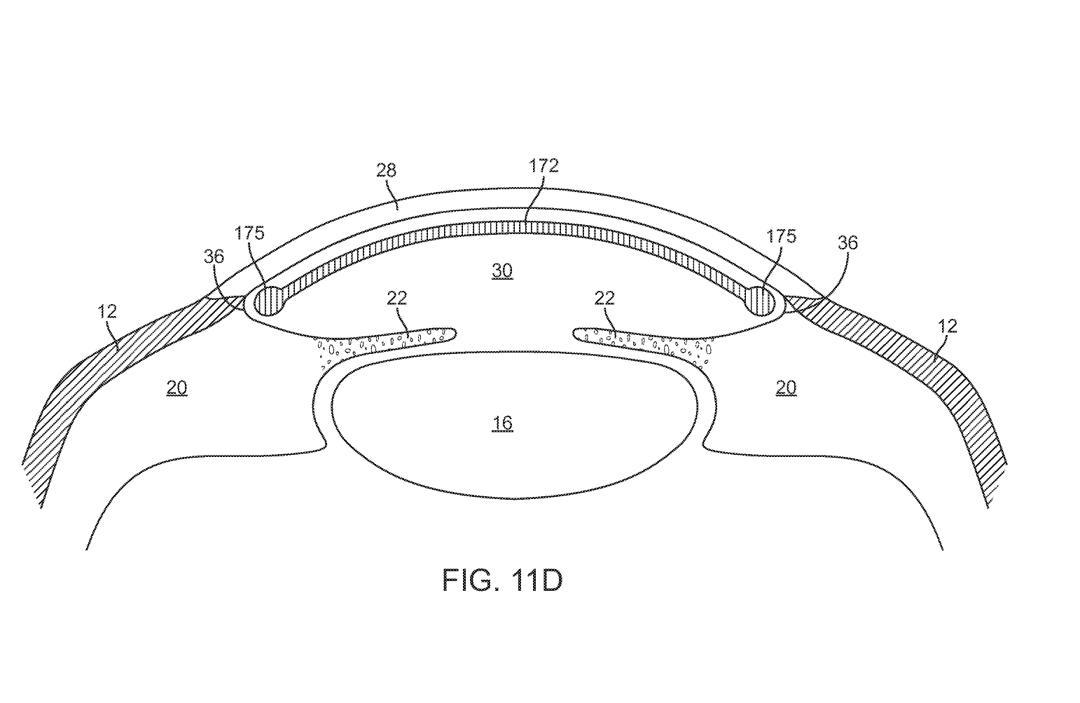

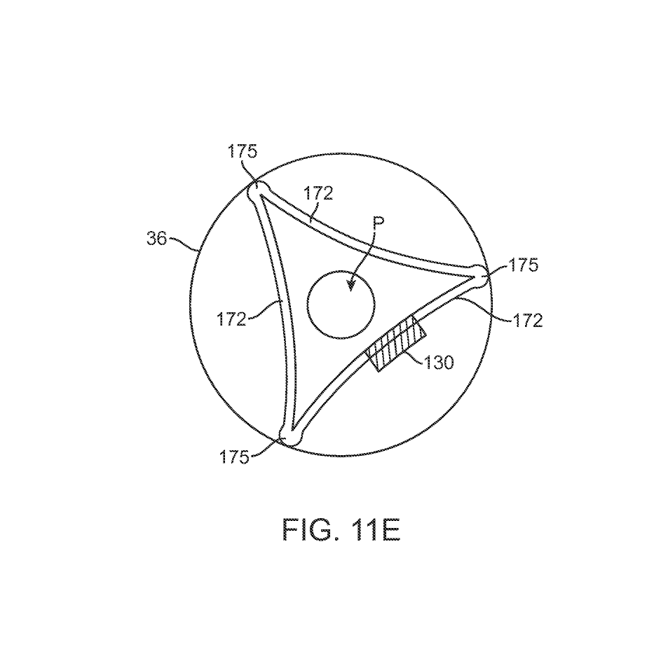

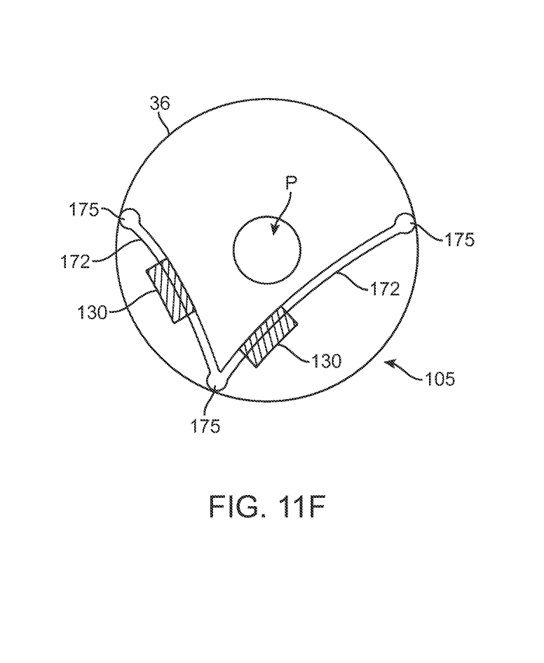

FIGS. 11A-11F show another implementation of a device configured to be implanted within the anterior chamber 30 to deliver one or more therapeutic agents from one or more reservoirs 130. The one or more reservoirs 130 can be formed according to any of a variety of configurations as described herein. For example, the reservoir 130 can include a penetrable surface or access port, one or more outlets, and one or more porous structures positioned relative to the outlets as described herein. The one or more reservoirs 130 can be coupled to a flexible scaffold 170 configured to fold, bend, or otherwise contract to a minimally invasive size such that the entire device can be delivered to the anterior chamber 30 through a clear corneal incision such as a self-sealing incision or cataract incision as known to one skilled in the art. The scaffold 170 can include one or more elongate arms 172 coupled to each other and including one or more contact elements 175. The contact elements 175 can be configured to contact selected regions within the eye to aid in positioning and retention of the device 105 within the anterior chamber 30. For example, the contact elements 175 can be positioned within the iridocorneo angle of the eye, such as within a region of the trabecular meshwork 36, to retain the reservoir 130 in proper location in the anterior chamber 30. The contact elements 175 can be positioned such that they abut the meshwork and are not inserted through the meshwork or can be inserted through a region of the trabecular meshwork. Generally, the contact elements 175 are located along the scaffold 170 where the scaffold 170 undergoes a bend or where the arms 172 terminate. In some implementations, the contact elements 175 make contact with at least three regions within the anterior chamber 30. The contact elements 175 can be atraumatic regions that are moderately enlarged in cross-sectional size compared to the cross-sectional diameter of the elongate arms 172 of the scaffold 170. This can allow the contact elements 175 to wedge within the angle of the eye near the trabecular meshwork 36 and provide stabilization of the device. The arms 172 of the scaffold 170 can vault away from the contact elements 175 such that the scaffold 170 does not contact any region of the eye, such as the iris 22, except where the contact elements 175 are wedged into the angle of the eye (see FIG. 11D). As such that scaffold 170 provides the device with an overall arched contour in the anterior chamber 30 when viewed in cross-section that conforms generally to the contour of the cornea 28. The arms 172 of the scaffold 170 can create any of a variety of shapes so long as the scaffold 170 remains outside the optical zone of the eye and avoids the pupil P (see FIGS. 11E and 11F). For example, the arms 172 of the scaffold 170 can form a triangular shape (see FIG. 11A), V-shape (see FIG. 11B), U-shape, S-shape, L-shape (see FIG. 11C) or other shape. It should be appreciated that the length of the arms 172 and the shape or relative arrangement of the arms 172 to one another can be customized prior to, during or after implantation. It should also be appreciated that the device can include more than a single reservoir 130 such as at least a second reservoir coupled to the scaffold 170. One or more of the elongate arms 172 can be coupled to one or more reservoirs 130 (see FIG. 11F). Further, it should be appreciated that the reservoir 130 can be formed by an elongate lumen extending through an interior of one or more of the arms 172 as shown in FIG. 11C. The lumen can be accessed via one or more injection ports 155 or penetrable locations such that the reservoir 130 can be filled or otherwise accessed after implantation. For example, an injection device can be inserted through a clear corneal incision to access the reservoir 130. As with other implementations described herein, the device 105 can include one or more outlets 135, such as near a terminus of the device, such that the therapeutic agents can exit the reservoir 130. In some implementations, the outlet 135 can be found on one of the contact elements 175. The device can also include one or more porous structures 150 positioned within the reservoir 130 such as near the outlet 135.

As mentioned above, the devices described herein can be configured to be refilled or flushed following implantation in the eye. Generally, the implementations of the devices described herein configured for refill contain drug solutions, drug suspensions and/or drug matrices. The devices described herein can also contain therapeutic agents formulated as one or more solid drug pellets formulated to deliver the one or more therapeutic agents at therapeutically effective amounts for an extended period of time. The period of time over which the device delivers therapeutically effective amounts can vary. In some implementations, the device is implanted to provide a therapy over the effective life of the device such that refill of the device is not necessary.

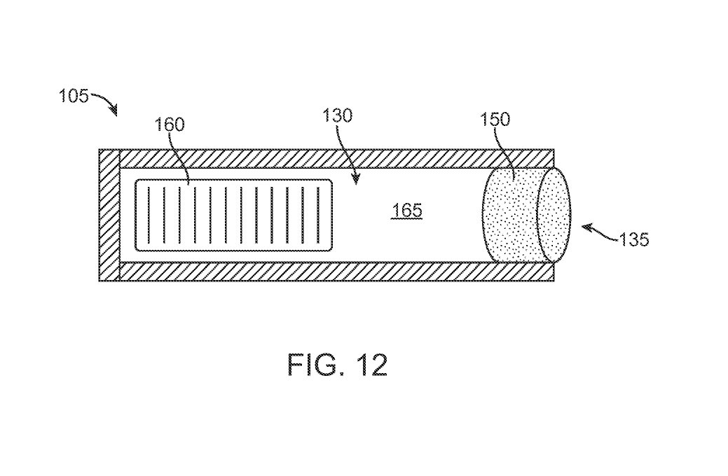

FIG. 12 shows a cross-sectional, side view of an implementation of a device 105 having a solid, non-permeable wall surrounding a reservoir 130 and configured to contain a solid pellet 160 of therapeutic material. The device 105 can have one or more fixation elements such as a flange element (not shown) or other type of fixation element as described herein. The device 105 can also include one or more outlets 135 in fluid communication with the reservoir 130. The outlet 135 can be covered by a porous structure 150 to control the release of material from the reservoir 130 into the eye also as described herein. A boundary layer of fluid 165 can separate the solid drug pellet 160 from the porous structure 150 at the outlet 135.

The size of the device, reservoir capacity, and location of implantation can be manipulated to increase duration of drug delivery from the device and as such the device 105 need not be refilled. The outer diameter of the device 105 can be sized such that it can be delivered using a 25 g needle. The length of the device can be between about 3 mm to about 7 mm. In some implementations, the reservoir 130 is formed of a 5 mm long polyimide tube having a wall thickness of 0.0127 mm and an outer diameter of approximately 0.52 mm. In some implementations, the volume of the reservoir 130 can be between about 0.2 uL to about 1 uL. In some implementations, the reservoir 130 can have a volume of 0.2 uL, contain 50% solid drug and provide 6 months of therapeutic delivery. In other implementations, the reservoir 130 can have a volume of 1 uL, contain a 50% solid drug core and provide 2.5 years of therapeutic delivery. A concentration of the drug in the fluid boundary layer 165 can be maintained at a solubility of the drug, e.g. the solubility of the prodrug (e.g. 300 mg/mL for bimatoprost).

Other Devices

FIG. 13A-13B illustrates another implementation of a device configured to deliver one or more therapeutic agents to the eye. The device 1305 can be a generally tubular implant having an internal lumen forming an internal drug reservoir 1330. The device 1305 can include one or more outlets 1335 such as near an end of the device to deliver the one or more therapeutic agents from the reservoir 1330 into a region of the eye such as the anterior chamber, the supraciliary space, the suprachoroidal space, Schlemm's Canal, the vitreous and other regions. In some implementations, the device 1305 can be positioned between tissue layers, such as for example a region of the ciliary body 20 and the sclera 12. The one or more outlets 1335 can be positioned such that they are located within the anterior chamber 30 near the angle of the eye to deliver material into the anterior chamber. Alternatively, the one or more outlets 1335 can be positioned such that they are located within the space between the tissue layers such as in the supraciliary or suprachoroidal spaces. In some implementations, the device 1305 can be anchored in place using one or more anchor elements (not shown). The anchor element can be an enlarged region of the device such as a flange or other element to provide anchoring of positioning of the device, such as within Schlemm's Canal 38.

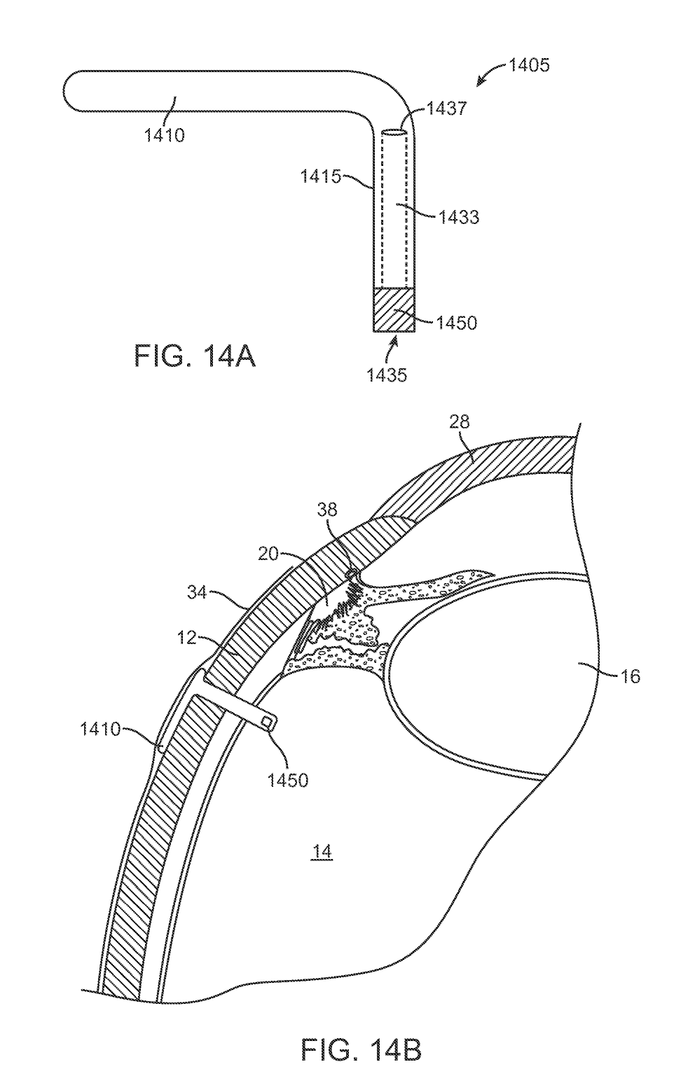

FIG. 14A-14B is a further implementation of a device 1405 for minimally invasive implantation for the treatment of the eye. The device 1405 can allow for drainage of aqueous out of the eye such as for the purpose of lowering intraocular pressure. In some implementations, the device 1405 includes a plate element or flange element 1410 coupled to a proximal end of a post 1415. The post 1415 can include an internal lumen 1433 in fluid communication with the flange element 1410 at a proximal opening 1437. The device 1405 can also include a distal opening 1435 or other openings along a region of the post 1415 that are in communication with the lumen 1433. The device 1405 can also include one or more porous structures 1450 positioned along the internal lumen 1433 such as near a distal opening 1435. The porous structure 1450 can mediate the drainage of the vitreous or aqueous from the eye towards the flange element 1410. The flange element 1410 can be positioned under the conjunctiva such that fluid exiting the eye can be directed to a subconjunctival region of the eye.

Delivery System and Methods of Implantation and Use

Described herein are a variety of devices used for the treatment of a patient. The implantation method of the devices described herein can vary depending on the type of device being implanted and the intended location and drug for treatment. The devices can be implanted using a delivery device having a tubular element 205 that is configured for minimally invasive implantation. For example, the tubular element 205 can have an outer diameter that is approximately 25 G or 0.5 mm or less. The tubular element 205 can include an internal volume within which the device 105 can be positioned extending between a proximal end region and a distal end region. The proximal end region can be coupled to an actuation mechanism such that the tubular element 205 can be withdrawn proximally to reveal the device 105 following implantation in the eye. The distal end region of the tubular element can have a distal tip configured to penetrate one or more eye tissues. In some implementations, the distal tip is beveled or sharpened such that it can be used to penetrate one or more tissues of the eye, such as the sclera through to the vitreous or the cornea through to the anterior chamber. The delivery device can incorporate another deployment structure that maintain the position of the device following implantation in the tissue, such as for example a stylet that can contact a proximal end of the device 105 such as the flange element 110 to maintain the device in position while the tubular element 205 is withdrawn in a proximal direction. It should also be appreciated that the device 105 can be positioned relative to the delivery device such that the distal end of the device 105 is exposed and configured to penetrate the eye tissue during delivery. For example, the distal end of the body 115 can be sharpened such that it can penetrate one or more eye tissues during delivery or be used to tunnel through or between eye tissues.

In one implementation of device implantation, a sclerotomy is created according to conventional techniques. The sclerotomy can be created posterior to an insertion site of the body 115 through the sclera 12 or the sclerotomy can be created directly above the insertion site of the post through the sclera 12. The conjunctiva 34 can be dissected and retracted so as to expose an area of the sclera 12. An incision in the conjunctiva 34 can be made remote from the intended insertion site of the device 105. A scleral incision or puncture can be formed. The scleral incision or puncture can be made with a delivery device tool or using a distal tip of the device, as described above. In some implementations, the device is implanted using sutureless surgical methods and devices. In other implementations, the device can be positioned sub-sclerally such as under a scleral flap. The post can be inserted into the eye (such as within the vitreous or the anterior chamber, etc.) until at least one of the outlets is positioned within or near the target delivery site and if a flange element is present until the inner-facing surface of the flange element can abut an outer surface of the eye. If a shape changing fixation element is incorporated, the elements can be held in a restrained configuration and released upon delivery to the implantation site such that the element can deform and provide fixation. An additional fixation element can be used such as a suture or other element if needed following implantation of the device in the eye. The device can remain in position to deliver the one or more therapeutic agents to the eye for a period of time including, but not limited to 1, 2, 3, 4, 5, 10, 15, 20, 25 days or any number of days, months and year, up to at least about 3 years. After the therapeutic agent has been delivered for the desired period of time, the device can be refilled for further delivery or removed.

Indications

In some implementations, the devices described herein are configured to be used to treat and/or prevent glaucoma. The devices described herein can cause a change in intraocular pressure that is between about 9 mmHg to about 15 mmHg. The target range in intraocular pressure is between about 8 mmHg or 30+% over baseline IOP reduction, such as for example 35-60%. The devices described herein provide a less invasive placement compared to more invasive procedures such as trabeculectomy.

The devices described herein can be used to treat and/or prevent a variety of other ocular conditions besides glaucoma, including but not limited to dry or wet age-related macular degeneration (AMD), neuroprotection of retinal ganglion cells, cataract or presbyopia prevention, cancers, angiogenesis, neovascularization, choroidal neovascularization (CNV) lesions, retinal detachment, proliferative retinopathy, proliferative diabetic retinopathy, degenerative disease, vascular diseases, occlusions, infection caused by penetrating traumatic injury, endophthalmitis such as endogenous/systemic infection, post-operative infections, inflammations such as posterior uveitis, retinitis or choroiditis and tumors such as neoplasms and retinoblastoma. Still further conditions that can be treated and/or prevented using the devices and methods described herein, include but are not limited to hemophilia and other blood disorders, growth disorders, diabetes, leukemia, hepatitis, renal failure, HIV infection, hereditary diseases such as cerebrosidase deficiency and adenosine deaminase deficiency, hypertension, septic shock, autoimmune diseases such as multiple sclerosis, Graves' disease, systemic lupus erythematosus and rheumatoid arthritis, shock and wasting disorders, cystic fibrosis, lactose intolerance, Crohn's disease, inflammatory bowel disease, gastrointestinal or other cancers, degenerative diseases, trauma, multiple systemic conditions such as anemia.

Therapeutics

Examples of therapeutic agents that may be delivered by the devices described herein are listed in Table 1 below.

In some implementations, prostaglandin analogues (PGAs) can be used to increase outflow of aqueous through the ciliary body and/or the trabecular meshwork. Because PGAs have the potential for CME complication with retinal exposure, it may be desirable to use them preferentially to target the anterior chamber rather than the vitreous. Drugs in this class include travaprost (0.004%), bimatoprost (0.03%, 0.01%), tafluprost (0.0015%), and latanoprost (0.005%). Beta blockers can be used to reduce aqueous fluid production by the ciliary body. Drugs in this class include timolol (0.5%). Carbonic anhydrase inhibitors can be used to reduce aqueous fluid production by the ciliary body as well. Drugs in this class include brinzolamide (1%), methazolamide, dorzolamide (2%), and acetazolamide. Alpha antagonists can be used to reduce aqueous fluid production by the ciliary body and increase outflow through the trabecular meshwork. Thus, the drug targets tissues located in both the anterior chamber and the posterior chamber and as such the devices can be implanted in either location to achieve a therapeutic result. Drugs in this class include brimonidine (0.1%, 0.15%) and apraclonidine (0.5%, 1.0%). Commercially available combinations of therapeutics considered herein include COMBIGAN® (brimonidine tartrate/timolol maleate ophthalmic solution; Allergan), and COSOPT® (dorzolamide hydrochloride-timolol maleate ophthalmic solution; Merck). Further, other sustained release therapeutics considered herein include subconjunctival latanoprost (Psivida/Pfizer), intracameral bimatoprost (Allergan), and intravitreal brimonidine (Allergan).

Other therapeutics that can be delivered from the devices described herein include but are not limited to Triamcinolone acetonide, Bimatoprost (Lumigan) or the free acid of bimatoprost, latanoprost or the free acid or salts of the free acid of latanoprost, Ranibizumab (Lucentis™), Travoprost (Travatan, Alcon) or the free acid or salts of the free acid of travoprost, Timolol (Timoptic, Merck), Levobunalol (Betagan, Allergan), Brimonidine (Alphagan, Allergan), Dorzolamide (Trusopt, Merck), Brinzolamide (Azopt, Alcon). Additional examples of therapeutic agents that may be delivered by the therapeutic device include antibiotics such as tetracycline, chlortetracycline, bacitracin, neomycin, polymyxin, gramicidin, cephalexin, oxytetracycline, chloramphenicol kanamycin, rifampicin, ciprofloxacin, tobramycin, gentamycin, erythromycin and penicillin; antifungals such as amphotericin B and miconazole; anti-bacterials such as sulfonamides, sulfadiazine, sulfacetamide, sulfamethizole and sulfisoxazole, nitrofurazone and sodium propionate; antivirals such as idoxuridine, trifluorotymidine, acyclovir, ganciclovir and interferon; antiallergenics such as sodium cromoglycate, antazoline, methapyriline, chlorpheniramine, pyrilamine, cetirizine and prophenpyridamine; anti-inflammatories such as hydrocortisone, hydrocortisone acetate, dexamethasone, dexamethasone 21-phosphate, fluocinolone, medrysone, prednisolone, prednisolone 21-phosphate, prednisolone acetate, fluoromethalone, betamethasone, and triamcinolone; non-steroidal anti-inflammatories such as salicylate, indomethacin, ibuprofen, diclofenac, flurbiprofen and piroxicam; decongestants such as phenylephrine, naphazoline and tetrahydrozoline; miotics and anticholinesterases such as pilocarpine, salicylate, acetylcholine chloride, physostigmine, eserine, carbachol, diisopropyl fluorophosphate, phospholine iodide and demecarium bromide; mydriatics such as atropine sulfate, cyclopentolate, homatropine, scopolamine, tropicamide, eucatropine and hydroxyamphetamine; sypathomimetics such as epinephrine; antineoplastics such as carmustine, cisplatin and fluorouracil; immunological drugs such as vaccines and immune stimulants; hormonal agents such as estrogens, estradiol, progestational, progesterone, insulin, calcitonin, parathyroid hormone and peptide and vasopressin hypothalamus releasing factor; beta adrenergic blockers such as timolol maleate, levobunolol HCl and betaxolol HCl; growth factors such as epidermal growth factor, fibroblast growth factor, platelet derived growth factor, transforming growth factor beta, somatotropin and fibronectin; carbonic anhydrase inhibitors such as dichlorophenamide, acetazolamide and methazolamide and other drugs such as prostaglandins, antiprostaglandins and prostaglandin precursors. Other therapeutic agents known to those skilled in the art which are capable of controlled, sustained release into the eye in the manner described herein are also suitable for use in accordance with embodiments of the claimed subject matter.

The therapeutic agent can also include one or more of the following: Abarelix, Abatacept, Abciximab, Adalimumab, Aldesleukin, Alefacept, Alemtuzumab, Alpha-1-proteinase inhibitor, Alteplase, Anakinra, Anistreplase, Antihemophilic Factor, Antithymocyte globulin, Aprotinin, Arcitumomab, Asparaginase, Basiliximab, Becaplermin, Bevacizumab, Bivalirudin, Botulinum Toxin Type A, Botulinum Toxin Type B, Capromab, Cetrorelix, Cetuximab, Choriogonadotropin alfa, Coagulation Factor IX, Coagulation factor VIIa, Collagenase, Corticotropin, Cosyntropin, Cyclosporine, Daclizumab, Darbepoetin alfa, Defibrotide, Denileukin diftitox, Desmopressin, Dornase Alfa, Drotrecogin alfa, Eculizumab, Efalizumab, Enfuvirtide, Epoetin alfa, Eptifibatide, Etanercept, Exenatide, Felypressin, Filgrastim, Follitropin beta, Galsulfase, Gemtuzumab ozogamicin, Glatiramer Acetate, Glucagon recombinant, Goserelin, Human Serum Albumin, Hyaluronidase, Ibritumomab, Idursulfase, Immune globulin, Infliximab, Insulin Glargine recombinant, Insulin Lyspro recombinant, Insulin recombinant, Insulin, porcine, Interferon Alfa-2a, Recombinant, Interferon Alfa-2b, Recombinant, Interferon alfacon-1, Interferonalfa-n1, Interferon alfa-n3, Interferon beta-1b, Interferon gamma-1b, Lepirudin, Leuprolide, Lutropin alfa, Mecasermin, Menotropins, Muromonab, Natalizumab, Nesiritide, Octreotide, Omalizumab, Oprelvekin, OspA lipoprotein, Oxytocin, Palifermin, Palivizumab, Panitumumab, Pegademase bovine, Pegaptanib, Pegaspargase, Pegfilgrastim, Peginterferon alfa-2a, Peginterferon alfa-2b, Pegvisomant, Pramlintide, Ranibizumab, Rasburicase, Reteplase, Rituximab, Salmon Calcitonin, Sargramostim, Secretin, Sermorelin, Serum albumin iodonated, Somatropin recombinant, Streptokinase, Tenecteplase, Teriparatide, Thyrotropin Alfa, Tositumomab, Trastuzumab, Urofollitropin, Urokinase, or Vasopressin. The molecular weights of the molecules and indications of these therapeutic agents are set for below in Table 1, below.

The therapeutic agent can include one or more of compounds that act by binding members of the immunophilin family of cellular proteins. Such compounds are known as “immunophilin binding compounds” Immunophilin binding compounds include but are not limited to the “limus” family of compounds. Examples of limus compounds that may be used include but are not limited to cyclophilins and FK506-binding proteins (FKBPs), including sirolimus (rapamycin) and its water soluble analog SDZ-RAD, tacrolimus, everolimus, pimecrolimus, CCI-779 (Wyeth), AP23841 (Ariad), and ABT-578 (Abbott Laboratories). The limus family of compounds may be used in the compositions, devices and methods for the treatment, prevention, inhibition, delaying the onset of, or causing the regression of angiogenesis-mediated diseases and conditions of the eye, including choroidal neovascularization. The limus family of compounds may be used to prevent, treat, inhibit, delay the onset of, or cause regression of AMD, including wet AMD. Rapamycin may be used to prevent, treat, inhibit, delay the onset of, or cause regression of angiogenesis-mediated diseases and conditions of the eye, including choroidal neovascularization. Rapamycin may be used to prevent, treat, inhibit, delay the onset of, or cause regression of AMD, including wet AMD.

The therapeutic agent can include one or more of: pyrrolidine, dithiocarbamate (NF.kappa.B inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™) and other antibodies directed to the same target; pegaptanib (Macugen™); vitronectin receptor antagonists, such as cyclic peptide antagonists of vitronectin receptor-type integrins; .alpha.-v/.beta.-3 integrin antagonists; .alpha.-v/.beta.-1 integrin antagonists; thiazolidinediones such as rosiglitazone or troglitazone; interferon, including .gamma.-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; apoptosis inhibiting agents; Visudyne™, snET2 and other photo sensitizers, which may be used with photodynamic therapy (PDT); inhibitors of hepatocyte growth factor (antibodies to the growth factor or its receptors, small molecular inhibitors of the c-met tyrosine kinase, truncated versions of HGF e.g. NK4).

The therapeutic agent can include a combination with other therapeutic agents and therapies, including but not limited to agents and therapies useful for the treatment of angiogenesis or neovascularization, particularly CNV. Non-limiting examples of such additional agents and therapies include pyrrolidine, dithiocarbamate (NF.kappa.B inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibizumab (Lucentis™) and other antibodies directed to the same target; pegaptanib (Macugen™); vitronectin receptor antagonists, such as cyclic peptide antagonists of vitronectin receptor-type integrins; .alpha.-v/.beta.-3 integrin antagonists; .alpha.-v/.beta.-1 integrin antagonists; thiazolidinediones such as rosiglitazone or troglitazone; interferon, including .gamma-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; inhibitors of hepatocyte growth factor (antibodies to the growth factor or its receptors, small molecular inhibitors of the c-met tyrosine kinase, truncated versions of HGF e.g. NK4); apoptosis inhibiting agents; Visudyne™, snET2 and other photo sensitizers with photodynamic therapy (PDT); laser photocoagulation; Pazopanib (Votrient™).

Formulations

As mentioned above, the devices described herein can be implanted using an incision or opening sized no greater than 0.5 mm. Further, upon implantation the devices described herein preferably avoid impacting the optical zone, the central visual axis and/or the optic axis. As such, the overall size and thus, the reservoir volume of the devices are limited. The one or more therapeutic agents delivered from the reservoir are formulated to allow for the greatest amount of drug in the least amount of volume such that they can be delivered for the longest duration of time. Fortunately, many of the current medications for the treatment of various eye conditions, such as glaucoma, are potent small molecules that require significantly smaller payloads for local delivery of the therapeutic agent.

In some implementations, the therapeutic agent to be delivered using the devices described herein is bimatoprost, latanoprost or another prostaglandin analogue. Latanoprost is an ester prodrug that penetrates into the eye after topical delivery and is rapidly hydrolyzed to the more potent free acid metabolite by esterases. Bimatoprost is an amide that is active in both its amide form and its free acid form, although the free acid does not penetrate into the eye. Solution formulations of, for example, latanoprost can be dissolved in concentrations higher than the solubility of the prodrug in water at pH 7. A higher concentration can be achieved by using formulations of the free acid and/or by the addition of one or more solubilizers, e.g. cyclodextrins, PEG, ethanol and others. Further, the formulations of the free acid will have a higher solubility in aqueous formulations than the parent prodrug. The free acid is the “active” component of the molecule and so can be used directly in the eye where it would otherwise be not active if applied topically to the surface of the eye since the free acid does not penetrate into the eye. The free acid delivered directly into the vitreous, however, would be active since this is the active form of the drug. Thus, a suspension of bimatoprost or latanoprost free acid (e.g. mixed with a silica gel) can be used to improve solubility and thus, the level of drug delivered to the vitreous. The drug suspension can also allow for a greater duration of treatment because more drug can be formulated in the suspension. For example, the solubility of bimatoprost in PBS at pH 7 is 300 ug/ml and the solubility of latanoprost in PBS at pH 7 is only 50 ug/ml, while the solubility of latanoprost free acid in PBS at pH 7 is 800 ug/ml. The free acid of bimatoprost, since it is a solid, can also be formulated as a biodegradable pellet. In addition, salts of other PGA free acids are known to be solids (see for example, US Patent Publication no. 2010-0105775, which is incorporated by reference herein) and can also be formulated as solid pellets or incorporated into formulations as suspensions.

Bimatoprost (or another similar drug) can be formulated in a solution-based payload where the free acid is used (rather than the prodrug) or solubilizing agents are added to the formulation. If the payload is a suspension or a predominantly solid drug form, the size of the reservoir can be even further reduced. For example, the volume of the reservoir can be as little as 1 uL if filled with a 10% suspension of bimatoprost while still providing up to 6 months of therapeutic delivery. Experimental modeling has shown that bimatoprost can be delivered at an effective therapeutic amount for 3-6 months from a reservoir of less than a 5 microliter volume. Target delivery rates of 40 ng/day to 300 ng/day of a solution of bimatoprost for a delivery duration of approximately 3 months can be achieved from the devices described herein where the fill concentration less than 15 mg/mL, the implant reservoir volume is between about 0.005 mL to about 0.010 mL, and the release rate index of the porous structure is between about 0.0013 to about 0.003 mm. Target delivery rates of 40 ng/day to 300 ng/day of a suspension of bimatoprost for a delivery duration of approximately 6 months can be achieved where the fill concentration is less than 20 mg/mL, the reservoir volume of the device is between about 0.005 mL to about 0.010 mL, and the release rate index of the porous structure is between about 0.003 mm to about 0.024 mm. Similarly, brimonidine, which has a solubility of about 600 ug/ml at pH 7, can be delivered for extended periods of time using similar devices. Description and calculation of the release rate index for a porous structure is described for example in for example U.S. Pat. No. 8,277,830, which is incorporated by reference herein.

In some aspects, the formulations of the current disclosure can be formulated to achieve high concentration (about 1 mg/mL-about 300 mg/mL) of a therapeutic agent, which is characterized as being not soluble in water or is poorly soluble in water.

In some aspects, the present disclosure provides formulations of a therapeutic agent, e.g., pazopanib or a pharmaceutically acceptable salt thereof, where the concentration in the device and/or at the target upon delivery can be between about 10 mg/mL to up to about 70 mg/mL (e.g., about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, or about 70 mg/mL). In some aspects, about 30 mg/mL to about 50 mg/mL of pazopanib in the formulation is provided.

In some aspects, the measured concentration is between about 10 mg/mL to up to about 70 mg/mL (e.g., about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, or about 70 mg/mL).

In some aspects, the fill concentration of the therapeutic agent, e.g., pazopanib or a pharmaceutically acceptable salt thereof, in the delivery device is between about 10 mg/mL to up to about 70 mg/mL (e.g., about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, or about 70 mg/mL).

In some aspects, the formulation includes a complexing agent, for example, sulfobutyl ether-β-cyclodextrin (“SBEβCD”) or CAPTISOL®.

In some aspects, additional components of the formulation, for example, without being a limiting example, are: trehalose, methylcellulose, ethylcellulose, sodium carboxymethylcellulose, hydroxypropylmethylcellulose, sodium hyaluronate, sodium alginate, chitosan and its derivatives, polyethylene glycol, glycerin, propylene glycol, Triacetin, N,N-Dimethylacetamide, pyrrolidone, dimethyl sulfoxide, ethanol, N-(-beta-Hydroxyethyl)-lactamide, 1-Methyl-2-pyrrolidinone, triglycerides, monothioglycerol, sorbitol, lecithin, methylparaben, propylparaben, polysorbates, block copolymers of ethylene oxide and propylene oxide, di-block polymers or tri-block copolymers of polyethylene oxide and polypropylene oxide, ethoxylated emulsifiers, polyethylene glycol esters, sucrose laurate, Tocopherol-PEG-succinate, phospholipids and their derivatives, or other non-ionic self-emulsifying agents.

In some aspects, solubilizing agents in the formulation of the current disclosure include, for example, without being a limiting example, trehalose, methylcellulose, ethylcellulose, sodium carboxymethylcellulose, sodium hyaluronate, sodium alginate, polyethylene glycol, glycerin, propylene glycol, Triacetin, N,N-Dimethylacetamide, poly(vinyl pyrrolidone), pyrrolidone, or combinations thereof. The solubilizing agent used in the preparation of formulations of the present disclosure is poly(vinyl pyrrolidone) (PVP). For example, the formulations of the current disclosure comprise between about 0.2% to about 1% PVP. The present disclosure provides formulations with between about 5 mg/mL PVP to about 30 mg/mL PVP.

Additional additives for including in the formulations of the present disclosure, for example, without being a limiting example, are triacetine (about 1× molar ration to the therapeutic agent), L-Lysine (about 25 mg/mL), ammonium acetate about 0.1%-about 5% (w/v) (e.g., about 2% (w/v)), or glycerol about 0.1%-about 5% (w/v) (e.g., about 2% (w/v)).

The formulation of the current disclosure can include one or two agents for pH adjustment for increasing buffering capacity of the formulation in the therapeutic device. One or two pH adjustment agents is/are selected from, without being a limiting example, sodium hydroxide, hydrochloric acid, citric acid, malic acid, acetate, tartaric acid, histidine, phosphate, or combinations thereof. In one embodiment, the formulation comprises agents for pH adjustment, but no complexing agents. The one or two pH adjusting agents are citric acid and/or histidine.

The formulation can include a tonicity adjusting agent. For example, the tonicity adjusting agent is, without being a limiting example, sodium chloride, sodium phosphate, or combinations thereof.

The formulations can have high stability during the use time of the PDS implant. For example, formulations can be stable in the reservoir chamber at 37° C. at physiological conditions for at least 6 months. For example, the formulations can be stable in the device in the presence of vitreous components diffusing from the vitreous.

The formulations can be used in a method of ocular drug delivery. The formulations of the present disclosure can be intravitreal delivery formulations or anterior chamber delivery formulations or posterior chamber delivery formulations. The formulations of the present disclosure are not formulated as eye drops. The formulations of the present disclosure are not formulated for topical delivery. The formulations of the present disclosure are not formulated for oral delivery or parenteral delivery. The formulations of the present disclosure are not formulated for periocular delivery.

Other pharmaceutically acceptable carriers for the therapeutic agents described herein can include such as, for example, solids such as starch, gelatin, sugars, natural gums such as acacia, sodium alginate and carboxymethyl cellulose; polymers such as silicone rubber; liquids such as sterile water, saline, dextrose, dextrose in water or saline; condensation products of castor oil and ethylene oxide, liquid glyceryl triester of a lower molecular weight fatty acid; lower alkanols; oils such as corn oil, peanut oil, sesame oil, castor oil, and the like, with emulsifiers such as mono- or di-glyceride of a fatty acid, or a phosphatide such as lecithin, polysorbate 80, and the like; glycols and polyalkylene glycols; aqueous media in the presence of a suspending agent, for example, sodium carboxymethylcellulose, sodium hyaluronate, sodium alginate, poly(vinyl pyrrolidone) and similar compounds, either alone, or with suitable dispensing agents such as lecithin, polyoxyethylene stearate and the like. The carrier may also contain adjuvants such as preserving, stabilizing, wetting, emulsifying agents or other related materials.

Materials

Generally, the components of the devices described herein are fabricated of materials that are biocompatible and preferably insoluble in the body fluids and tissues that the device comes into contact with. The materials generally do not cause irritation to the portion of the eye that it contacts. Materials may include, by way of example, various polymers including, for example, silicone elastomers and rubbers, polyolefins, polyurethanes, acrylates, polycarbonates, polyamides, polyimides, polyesters, and polysulfones. One or more components of the devices described herein can be fabricated of a permeable material including, but not limited to, polycarbonates, polyolefins, polyurethanes, copolymers of acrylonitrile, copolymers of polyvinyl chloride, polyamides, polysulphones, polystyrenes, polyvinyl fluorides, polyvinyl alcohols, polyvinyl esters, polyvinyl butyrate, polyvinyl acetate, polyvinylidene chlorides, polyvinylidene fluorides, polyimides, polyisoprene, polyisobutylene, polybutadiene, polyethylene, polyethers, polytetrafluoroethylene, polychloroethers, polymethylmethacrylate, polybutylmethacrylate, polyvinyl acetate, nylons, cellulose, gelatin, silicone rubbers and porous rubbers. One or more components of the devices described herein can be fabricated of a nonbiodegradable polymer, including but not limited to polymethylmethacrylate, a silicone elastomer, or silicone rubber. Other suitable non-erodible, biocompatible polymers which may be used in fabricating the devices described herein may include polyolefins such as polypropylene and polyethylene, homopolymers, and copolymers of vinyl acetate such as ethylene vinyl acetate copolymer, polyvinylchlorides, homopolymers and copolymers of acrylates such as polyethylmethacrylate, polyurethanes, polyvinylpyrrolidone, 2-pyrrolidone, polyacrylonitrile butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acrylonitrile butadiene styrene, polymethylpentene, polysulfones, polyesters, polyimides, natural rubber, polyisobutylene, polymethylstyrene and other similar non-erodible biocompatible polymers.

One or more of the components of the devices described herein can be fabricated of a rigid, non-pliable material. One or more of the components of the devices described herein can be fabricated of a shape memory material and/or superelastic material including, but not limited to shape memory alloys (SMA) like nitinol (Ni—Ti alloy) and shape memory polymers (SMP) like AB-polymer networks based on oligo(e-caprolactone) dimethacrylates and n-butyl acrylate. Shape memory alloys generally have at least two phases: (1) a martensite phase, which has a relatively low tensile strength and which is stable at relatively low temperatures, and (2) an austenite phase, which has a relatively high tensile strength and which is stable at temperatures higher than the martensite phase. The shape memory characteristics are imparted on the material by heating the material to a temperature above the temperature at which the austenite phase is stable. While the material is heated to this temperature, the device is held in the “memory shape”, which is shape that is desired to be “remembered”.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed. The foregoing description is intended to illustrate and not limit the scope of the claimed subject matter, which is defined by the scope of the appended claims.

TABLE 1
List of Therapeutic Agent Examples

| | | | | Molecular |
|---|---|---|---|---|
| Generic Name | Brands (Companies) | Category | Indication | Weight |
| 2-Methoxyestradiol | (Paloma | Angiogenesis inhibitors | AMD | |
| analogs | Pharmaceuticals) | | | |
| 3-aminothalidomide | | | | |
| 13-cis retinoic acid | Accutane TM (Roche | | | |
| | Pharmaceuticals) | | | |
| A0003 | (Aqumen | A0003 | AMD | |
| | BioPharmaceuticals) | | | |
| A5b1 integrin inhibitor | (Jerini Ophthalmic); | Inhibitors of a5b1 integrin | AMD | |
| | (Ophthotech) | | | |
| Abarelix | Plenaxis ™ (Praecis | Anti-Testosterone Agents; | For palliative treatment of | 37731 |
| | Pharmaceuticals) | Antineoplastic Agents | advanced prostate cancer. | |
| Abatacept | Orencia ™ (Bristol- | Antirheumatic Agents | For the second line reduction of | 37697 |
| | Myers Squibb) | | the signs and symptoms of | |
| | | | moderate-to-severe active | |
| | | | rheumatoid arthritis, inducing | |
| | | | major clinical response, slowing | |
| | | | the progression of structural | |
| | | | damage, and improving physical | |
| | | | function in adult patients who | |
| | | | have | |
| [symbol] bciximab | ReoPro ™; ReoPro ™ | Anticoagulants; | For treatment of myocardial | 42632 |
| | (Centocor) | Antiplatelet Agents | infarction, adjunct to | |
| | | | percutaneous coronary | |
| | | | intervention, unstable angina | |
| ABT-578 | (Abbott Laboratories) | Limus Immunophilin | | |
| | | Binding Compounds | | |
| [symbol] cetonide | | | | |
| [symbol] dalimumab | Humira ™ (Abbott | Antirheumatic Agents; | Uveitis, AMD | 25645 |
| | Laboratories) | Immunomodulatory Agents | | |
| Aldesleukin | Proleukin ™; | Antineoplastic Agents | For treatment of adults with | 61118 |
| | Proleukin ™ (Chiron | | metastatic renal cell carcinoma | |
| | Corp) | | | |
| [symbol] lefacept | Amevive ™ | Immunomodulatory | For treatment of moderate to | 42632 |
| | | Agents; | severe chronic plaque psoriasis | |
| | | Immunosuppressive | | |
| | | Agents | | |
| Alemtuzumab | Campath ™; Campath ™ | Antineoplastic Agents | For treatment of B-cell chronic | 6614 |
| | (ILEX Pharmaceuticals | | lymphocytic leukemia | |
| | LP); MabCampath ™ | | | |
| Alpha-1-proteinase | Aralast ™ (Baxter); | Enzyme Replacement | For treatment of panacinar | 28518 |
| inhibitor | Prolastin ™ (Talecris | Agents | emphysema | |
| | Biotherapeutics C | | | |
| | formerly Bayer) | | | |
| Alteplase | Activase ™ (Genentech | Thrombolytic Agents | For management of acute | 54732 |
| | Inc) | | myocardial infarction, acute | |
| | | | ischemic stroke and for lysis of | |
| | | | acute pulmonary emboli | |
| AMG-1470 | | | | |
| Anakinra | Kineret ™ (Amgen Inc) | Anti-Inflammatory Agents, | For the treatment of adult | 65403 |
| | | Non-Steroidal; Anti- | rheumatoid arthritis. | |
| | | rheumatic Agents; | | |
| | | Immunomodulatory Agents | | |
| Anecortave acetate | | | | |
| Angiostatin | | | | |
| Anistreplase | Eminase ™ (Wulfing | Thrombolytic Agents | For lysis of acute pulmonary | 54732 |
| | Pharma GmbH) | | emboli, intracoronary emboli and | |
| | | | management of myocardial | |
| | | | infarction | |
| Anti-angiogenesis | (Eyecopharm) | Anti-angiogenesis peptides | AMD | |
| peptides | | | | |
| Anti-angiogenesis | (TRACON Pharma) | Anti-angiogenesis | AMD | |
| antibodies, TRC093, | | antibodies | | |
| TRC105 | | | | |
| Anti-angiogeric | Icon-1 ™ (Iconic | Anti-angiogeric bifunctional | AMD | |
| bifunctional protein | Therapeutics) | protein, Icon-1 | | |
| Anti-endothelial growth | | | | |
| factor | | | | |
| Antihemophilic Factor | Advate ™; Alphanate ™; | Coagulants; Thrombotic | For the treatment of hemophilia | 70037 |
| | Bioclate ™; Helixate ™; | Agents | A, von Willebrand disease and | |
| | Helixate FS ™; Hemofil | | Factor XIII deficiency | |
| | M ™; Humate-P ™; | | | |
| | Hyate:C ™; Koate- | | | |
| | HP ™; Kogenate ™; | | | |
| | Kogenate FS ™; | | | |
| | Monarc-M ™; | | | |
| | Monoclate-P ™; | | | |
| | ReFacto ™; Xyntha ™ | | | |
| Antithymocyte globulin | Genzyme); | Immunomodulatory Agents | For prevention of renal | 37173 |
| | Thymoglobulin ™ | | transplant rejection | |
| | (SangStat Medical | | | |
| Anti-hypertensive | (MacuCLEAR) | Anti-hypertensive MC1101 | AMD | |
| MC1101 | | | | |
| Anti-platelet devired | | | | |
| growth factor | | | | |
| Anti-VEGF | (Neurotech); Avastin ™ | Anti-VEGF | AMD | |
| | (NeoVista) | | | |
| AP23841 | (Ariad) | Limus Immunophilin | | |
| | | Binding Compounds | | |
| ARC1905 | Ophthotech | Complement Cascade | | |
| | | Inhibitor (Factor C5) | | |
| Aprotinin | Trasylol ™ | Antifibrinolytic Agents | For prophylactic use to reduce | 90569 |
| | | | perioperative blood loss and the | |
| | | | need for blood transfusion in | |
| | | | patients undergoing | |
| | | | cardiopulmonary bypass in the | |
| | | | course of coronary artery bypass | |
| | | | graft surgery who are at an | |
| | | | increased risk for blood loss and | |
| | | | blood transfusion | |
| Arcitumomab | CEA-Scan ™ | Diagnostic Agents; | For imaging colorectal tumors | 57561 |
| | | Imaging Agents | | |
| Asparaginase | Elspar ™ (Merck & Co. | Antineoplastic Agents | For treatment of acute | 132.118 |
| | Inc) | | lymphocytic leukemia and non- | |
| | | | Hodgkins lymphoma | |
| Axitinib | | Tyrosine Kinase Inhibitors | | 386 |
| Basiliximab | Simulect ™ (Novartis | Immunomodulatory | For prophylactic treatment of | 61118 |
| | Pharmaceuticals) | Agents; | kidney transplant rejection | |
| | | Immunosuppressive | | |
| | | Agents | | |
| Becaplermin | Regranex ™; | Anti-Ulcer Agents; Topical | For topical treatment of skin | 123969 |
| | Regranex ™ (OMJ | | ulcers (from diabetes) | |
| | Pharmaceuticals) | | | |
| Bevacizumab | Avastin ™; Avastin ™ | Antiangiogenesis Agents; | For treatment of metastatic | 27043 |
| | (Genentech Inc) | Antineoplastic Agents | colorectal cancer | |
| Bivalirudin | Angiomax ™; | Anticoagulants; | For treatment of heparin-induced | 70037 |
| | Angiomax ™ (Medicines | Antithrombotic Agents | thrombocytopenia | |
| | Co or MDCO); | | | |
| | Angiox ™ | | | |
| Bortezomib | | Proteosome Inhibitors | | |
| Bosutinib | | Tyrosine Kinase Inhibitors | | 530 |
| Botulinum Toxin Type A | BOTOX ™ (Allegran | Anti-Wrinkle Agents; | For the treatment of cervical | 23315 |
| | Inc); BOTOX | Antidystonic Agents; | dystonia in adults to decrease | |
| | Cosmetic ™ (Allegran | Neuromuscular Blocking | the severity of abnormal head | |
| | Inc); Botox ™; | Agents | position and neck pain | |
| | Dysport ™ | | associated with cervical | |
| | | | dystonia. Also for the treatment | |
| | | | of severe primary axillary | |
| | | | hyperhidrosis that is | |
| | | | inadequately managed with | |
| | | | topical | |
| Botulinum Toxin Type B | Myobloc ™ (Solstice | Antidystonic Agents | For the treatment of patients with | 12902 |
| | Neurosciences); | | cervical dystonia to reduce the | |
| | Neurobloc ™ (Solstice | | severity of abnormal head | |
| | Neurosciences) | | position and neck pain | |
| | | | associated with cervical | |
| | | | dystonia. | |
| C5 inhibitor | (Jerini Ophthalmic); | Inhibitors of C5 | AMD | |
| | (Ophthotech) | | | |
| Cal101 | Calistoga | PI3Kdelta Inhibitor | AMD, DME | |
| Canstatin | | | | |
| Capromab | ProstaScint ™ (Cytogen | Imaging Agents | For diagnosis of prostate cancer | 84331 |
| | Corp) | | and detection of intra-pelvic | |
| | | | metastases | |
| Captopril | | ACE Inhibitors | | |
| CCI-779 | (Wyeth) | Limus Immunophilin | | |
| | | Binding Compounds | | |
| Cediranib | | Tyrosine Kinase Inhibitors | | 450 |
| Celecoxib | | Cyclooxygenase Inhibitors | | |
| Cetrorelix | Cetrotide ™ | Hormone Antagonists; | For the inhibition of premature | 78617 |
| | | Infertility Agents | LH surges in women undergoing | |
| | | | controlled ovarian stimulation | |
| Cetuximab | Erbitux ™; Erbitux ™ | Antineoplastic Agents | For treatment of metastatic | 42632 |
| | (ImClone Systems Inc) | | colorectal cancer. | |
| Choriogonadotropin alfa | Novarel ™; Ovidrel ™; | Fertility Agents; | For the treatment of female | 78617 |
| | Pregnyl ™; Profasi ™ | Gonadotropins | infertility | |
| Cilary neurotrophic factor | (Neurotech) | Cilary neurotrophic factor | AMD | |
| Coagulation Factor IX | Benefix ™ (Genetics | Coagulants; Thrombotic | For treatment of hemophilia | 267012 |
| | Institute) | Agents | (Christmas disease). | |
| [symbol] oagulation factor VIIa | NovoSeven ™ (Novo | Coagulants; Thrombotic | For treatment of hemorrhagic | 54732 |
| | Nordisk) | Agents | complications in hemophilia A | |
| | | | and B | |
| Colchicines | | | | |
| Collagenase | Cordase ™; Santyl ™ | Anti-Ulcer Agents; Topical | For treatment of chronic dermal | 138885 |
| | (Advance Biofactures | | ulcers and severe skin burns | |
| | Corp); Xiaflextm ™ | | | |
| Complement factor H | (Optherion); (Taligen | Complement factor H | AMD, Geographic Atrophy | |
| recombinant | Therapeutics) | recombinant | | |
| Compstatin derivative | (Potentia | Complement Factor C3 | AMD | |
| peptide, POT-4 | Pharmaceuticals) | Inhibitors; Compstatin | | |
| | | Derivative Peptides | | |
| Corticotropin | ACTH ™; Acethropan ™; | Diagnostic Agents | For use as a diagnostic agent in | 33927 |
| | Acortan ™; Acthar ™; | | the screening of patients | |
| | Exacthin ™; H.P. Acthar | | presumed to have adrenocortical | |
| | Gel ™; Isactid ™; | | insufficiency. | |
| | Purified cortrophin | | | |
| | gel ™; Reacthin ™; | | | |
| | Solacthyl ™; Tubex | | | |
| Cosyntropin | Cortrosyn ™; Synacthen | Diagnostic Agents | For use as a diagnostic agent in | 33927 |
| | depot ™ | | the screening of patients | |
| | | | presumed to have adrenocortical | |
| | | | insufficiency. | |
| Cyclophilins | | Limus Immunophilin | | |
| | | Binding Compounds | | |
| Cyclosporine | Gengraf ™ (Abbott | Antifungal Agents; | For treatment of transplant | 32953 |
| | labs); Neoral ™ | Antirheumatic Agents; | rejection, rheumatoid arthritis, | |
| | (Novartis); Restasis ™; | Dermatologic Agents; | severe psoriasis | |
| | Restasis ™ (Allergan | Enzyme Inhibitors; | | |
| | Inc); Sandimmune ™ | Immunomodulatory | | |
| | (Novartis); Sangcya ™ | Agents; | | |
| | | Immunosuppressive | | |
| | | Agents | | |
| Daclizumab | Zenapax ™ (Hoffmann- | Immunomodulatory | For prevention of renal | 61118 |
| | La Roche Inc) | Agents; | transplant rejection; Uveitis | |
| | | Immunosuppressive | | |
| | | Agents | | |
| Darbepoetin alfa | Aranesp ™ (Amgen | Antianemic Agents | For the treatment of anemia | 55066 |
| | Inc.) | | (from renal transplants or certain | |
| | | | HIV treatment) | |
| Dasatinib | | Tyrosine Kinase Inhibitors | | 488 |
| Defibrotide | Dasovas ™; Noravid ™; | Antithrombotic Agents | Defibrotide is used to treat or | 36512 |
| | Prociclide ™ | | prevent a failure of normal blood | |
| | | | flow (occlusive venous disease, | |
| | | | OVD) in the liver of patients who | |
| | | | have had bone marrow | |
| | | | transplants or received certain | |
| | | | drugs such as oral estrogens, | |
| | | | mercaptopurine, and many | |
| | | | others. | |
| Denileukin diftitox | Ontak ™ | Antineoplastic Agents | For treatment of cutaneous T- | 61118 |
| | | | cell lymphoma | |
| Desmopressin | Adiuretin ™; | Antidiuretic Agents; | For the management of primary | 46800 |
| | Concentraid ™; | Hemostatics; Renal | nocturnal enuresis and indicated | |
| | Stimate ™ | Agents | as antidiuretic replacement | |
| | | | therapy in the management of | |
| | | | central diabetes insipidus and for | |
| | | | the management of the | |
| | | | temporary polyuria and | |
| | | | polydipsia following head trauma | |
| | | | or surgery in the pitu | |
| Dexamethasone | Ozurdex ™ (Allergan) | Glucocorticoid | DME, inflammation, macular | 392 |
| | | | edema following branch retinal | |
| | | | vein occlusion (BRVO) or central | |
| | | | retinal vein occlusion (CRVO) | |
| Diclofenac | | Cyclooxygenase Inhibitors | | |
| Dithiocarbamate | | NFκB Inhibitor | | |
| Dornase Alfa | Dilor ™; Dilor-400 ™; | Enzyme Replacement | For the treatment of cystic | 7656 |
| | Lufyllin ™; Lufyllin- | Agents | fibrosis. | (double |
| | 400 ™; Neothylline ™; | | | strand) |
| | Pulmozyme ™ | | | |
| | (Genentech Inc) | | | |
| [symbol] rotrecogin alfa | Xigris ™; Xigris ™ (Eli | Antisepsis Agents | For treatment of severe sepsis | 267012 |
| | Lilly & Co) | | | |
| [symbol] culizumab | Soliris ™; Soliris ™ | Complement Cascade | AMD | 188333 |
| | (Alexion | Inhibitor (Factor C5) | | |
| | Pharmaceuticals) | | | |
| [symbol] falizumab | Raptiva ™; Raptiva ™ | Immunomodulatory | For the treatment of adult | 128771 |
| | (Genentech Inc) | Agents; | patients with moderate to severe | |
| | | Immunosuppressive | chronic plaque psoriasis, who | |
| | | Agents | are candidates for phototherapy | |
| | | | or systemic therapy. | |
| Endostatin | | | | |
| Enfuvirtide | Fuzeon ™; Fuzeon ™ | Anti-HIV Agents; HIV | For treatment of HIV AIDS | 16768 |
| | (Roche | Fusion Inhibitors | | |
| | Pharmaceuticals) | | | |
| Epoetin alfa | Epogen ™ (Amgen | Antianemic Agents | For treatment of anemia (from | 55066 |
| | Inc.); Epogin ™ | | renal transplants or certain HIV | |
| | (Chugai); Epomax ™ | | treatment) | |
| | (Elanex); Eprex ™ | | | |
| | (Janssen-Cilag. Ortho | | | |
| | Biologies LLC); | | | |
| | NeoRecormon ™ | | | |
| | (Roche); Procrit ™ | | | |
| | (Ortho Biotech); | | | |
| | Recormon ™ (Roche) | | | |
| Eptifibatide | Integrilin ™; Integrilin ™ | Anticoagulants; | For treatment of myocardial | 7128 |
| | (Millennium Pharm) | Antiplatelet Agents; | infarction and acute coronary | |
| | | Platelet Aggregation | syndrome. | |
| | | Inhibitors | | |
| Erlotinib | | Tyrosine Kinase Inhibitors | | 393 |
| [symbol] tanercept | Enbrel ™; Enbrel ™ | Antirheumatic Agents; | Uveitis, AMD | 25645 |
| | (Immunex Corp) | Immunomodulatory Agents | | |
| Everolimus | Novartis | Limus Immunophilin | AMD | |
| | | Binding Compounds, | | |
| | | mTOR | | |
| Exenatide | Byetta ™; Byetta ™ | | Indicated as adjunctive therapy | 53060 |
| | (Amylin/Eli Lilly) | | to improve glycemic control in | |
| | | | patients with Type 2 diabetes | |
| | | | mellitus who are taking | |
| | | | metformin, a sulfonylurea, or a | |
| | | | combination of both, but have | |
| | | | not achieved adequate glycemic | |
| | | | control. | |
| FCFD4514S | Genentech/Roche | Complement Cascade | AMD, Geographic Atrophy | |
| | | Inhibitor (Factor D) | | |
| Felypressin | Felipresina ™ [INN- | Renal Agents; | For use as an alternative to | 46800 |
| | Spanish]; | Vasoconstrictor Agents | adrenaline as a localizing agent, | |
| | Felipressina ™ [DCIT]; | | provided that local ischaemia is | |
| | Felypressin ™ | | not essential. | |
| | [USAN:BAN:INN]; | | | |
| | Felypressine ™ [INN- | | | |
| | French]; | | | |
| | Felypressinum ™ [INN- | | | |
| | Latin]; Octapressin ™ | | | |
| Fenretinide | Sirion/reVision | Binding Protein Antagonist | AMD, Geographic Atrophy | |
| | Therapeutics | for Oral Vitamin A | | |
| Filgrastim | Neupogen ™ (Amgen | Anti-Infective Agents; | Increases leukocyte production, | 28518 |
| | Inc.) | Antineutropenic Agents; | for treatment in non-myeloid | |
| | | Immunomodulatory Agents | cancer, neutropenia and bone | |
| | | | marrow transplant | |
| FK605-binding proteins, | | Limus Immunophilin | | |
| FKBPs | | Binding Compounds | | |
| Fluocinolone Acetonide | Retisert ™ (Bausch & | Glucocorticoid | Retinal inflammation, diabetic | 453 |
| | Lomb); Iluvien ™ | | macular edema | |
| | (Alimera Sciences, Inc.) | | | |
| Follitropin beta | Follistim ™ (Organon); | Fertility Agents | For treatment of female infertility | 78296 |
| | Gonal F ™; Gonal-F ™ | | | |
| Fumagillin | | | | |
| Falsulfase | Naglazyme ™; | Enzyme Replacement | For the treatment of adults and | 47047 |
| | Naglazyme ™ (BioMarin | Agents | children with | |
| | Pharmaceuticals) | | Mucopolysaccharidosis VI. | |
| Fefitinib | | Tyrosine Kinase Inhibitors | | 447 |
| Femtuzumab ozogamicin | Mylotarg ™; Mylotarg ™ | Antineoplastic Agents | For treatment of acute myeloid | 39826 |
| | (Wyeth) | | leukemia | |
| Flatiramer Acetate | Copaxone ™ | Adjuvants, Immunologic; | For reduction of the frequency of | 29914 |
| | | Immunosuppressive | relapses in patients with | |
| | | Agents | Relapsing-Remitting Multiple | |
| | | | Sclerosis. | |
| Glucagon recombinant | GlucaGen ™ (Novo | Antihypoglycemic Agents | For treatment of severe | 54009 |
| | Nordisk); Glucagon ™ | | hypoglycemia, also used in | |
| | (Eli Lilly) | | gastrointestinal imaging | |
| Goserelin | Zoladex ™ | Antineoplastic Agents; | Breast cancer; Prostate | 78617 |
| | | Antineoplastic Agents, | carcinoma; Endometriosis | |
| | | Hormonal | | |
| Human Serum Albumin | Albutein ™ (Alpha | Serum substitutes | For treatment of severe blood | 39000 |
| | Therapeutic Corp) | | loss, hypervolemia, | |
| | | | hypoproteinemia | |
| Hyaluronidase | Vitragan ™; Vitrase ™; | Anesthetic Adjuvants; | For increase of absorption and | 69367 |
| | Vitrase ™ (Ista Pharma) | Permeabilizing Agents | distribution of other injected | |
| | | | drugs and for rehydration | |
| Ibritumomab | Zevalin ™ (IDEC | Antineoplastic Agents | For treatment of non-Hodgkin's | 33078 |
| | Pharmaceuticals) | | lymphoma | |
| Idursulfase | Elaprase ™ (Shire | Enzyme Replacement | For the treatment of Hunter | 47047 |
| | Pharmaceuticals) | Agents | syndrome in adults and children | |
| | | | ages 5 and older. | |
| Imatinib | | Tyrosine Kinase Inhibitors | AMD, DME | 494 |
| Immune globulin | Civacir ™; | Anti-Infectives; | For treatment of | 42632 |
| | Flebogamma ™ | Immunomodulatory Agents | immunodeficiencies, | |
| | (Institute Grifols SA); | | thrombocytopenic purpura, | |
| | Gamunex ™ (Talecris | | Kawasaki disease, | |
| | Biotherapeutics) | | gammablobulinemia, leukemia, | |
| | | | bone transplant | |
| Infliximab | Remicade ™ (Centocor | Immunomodulatory | Uveitis, AMD | 25645 |
| | Inc) | Agents; | | |
| | | Immunosuppressive | | |
| | | Agents | | |
| Insulin Glargine | Lantus ™ | Hypoglycemic Agents | For treatment of diabetes (type I | 156308 |
| recombinant | | | and II) | |
| Insulin Lyspro | Humalog ™ (Eli Lily); | Hypoglycemic Agents | For treatment of diabetes (type I | 154795 |
| recombinant | Insulin Lispro (Eli Lily) | | and II) | |
| Insulin recombinant | Novolin R ™ (Novo | Hypoglycemic Agents | For treatment of diabetes (type I | 156308 |
| | Nordisk) | | and II) | |
| Insulin, porcine | Iletin II ™ | Hypoglycemic Agents | For the treatment of diabetes | 156308 |
| | | | (type I and II) | |
| Interferon | | | | |
| Interferon Alfa-2a, | Roferon A ™ | Antineoplastic Agents; | For treatment of chronic hepatitis | 57759 |
| recombinant | (Hoffmann-La Roche | Antiviral Agents | C, hairy cell leukemia, AIDS- | |
| | Inc); Veldona ™ | | related Kaposi's sarcoma, and | |
| | (Amarillo Biosciences) | | chronic myelogenous leukemia. | |
| | | | Also for the treatment of oral | |
| | | | warts arising from HIV infection. | |
| Interferon Alfa-2b, | Intron A ™ (Schering | Antineoplastic Agents; | For the treatment of hairy cell | 57759 |
| recombinant | Corp) | Antiviral Agents; | leukemia, malignant melanoma, | |
| | | Immunomodulatory Agents | and AIDS-related Kaposi's | |
| | | | sarcoma. | |
| Interferon alfacon-1 | Advaferon ™; | Antineoplastic Agents; | For treatment of hairy cell | 57759 |
| | Infergen ™ (InterMune | Antiviral Agents; | leukemia, malignant melanoma, | |
| | Inc) | Immunomodulatory Agents | and AIDS-related Kaposi's | |
| | | | sarcoma | |
| Interferon alfa-n1 | Wellferon ™ | Antiviral Agents; | For treatment of venereal or | 57759 |
| | (GlaxoSmithKline) | Immunomodulatory Agents | genital warts caused by the | |
| | | | Human Papiloma Virus | |
| Interferon alfa-n3 | Alferon ™ (Interferon | Antineoplastic Agents; | For the intralesional treatment of | 57759 |
| | Sciences Inc.); Alferon | Antiviral Agents; | refractory or recurring external | |
| | LDO ™; Alferon N | Immunomodulatory Agents | condylomata 47cuminate. | |
| | Injection ™ | | | |
| Interferon beta-1b | Betaseron ™ (Chiron | Antiviral Agents; | For treatment of | 57759 |
| | Corp) | Immunomodulatory Agents | relapsing/remitting multiple | |
| | | | sclerosis | |
| Interferon gamma-1b | Actimmune ™; | Antiviral Agents; | For treatment of Chronic | 37835 |
| | Actimmune ™ | Immunomodulatory Agents | granulomatous disease, | |
| | (InterMune Inc) | | Osteopetrosis | |
| Lapatinib | | Tyrosine Kinase Inhibitors | | 581 |
| Lepirudin | Refludan ™ | Anticoagulants; | For the treatment of heparin- | 70037 |
| | | Antithrombotic Agents; | induced thrombocytopenia | |
| | | Fibrinolytic Agents | | |
| Lestaurtinib | | Tyrosine Kinase Inhibitors | | 439 |
| Leuprolide | Eligard ™ (Atrix | Anti-Estrogen Agents; | For treatment of prostate cancer, | 37731 |
| | Labs/QLT Inc) | Antineoplastic Agents | endometriosis, uterine fibroids | |
| | | | and premature puberty | |
| Lutropin alfa | Luveris ™ (Serono) | Fertility Agents | For treatment of female infertility | 78617 |
| Mecasermin | Increlex ™; Increlex ™ | | For the long-term treatment of | 154795 |
| | (Tercica); Iplex | | growth failure in pediatric | |
| | | | patients with Primary IGFD or | |
| | | | with GH gene deletion who have | |
| | | | developed neutralizing | |
| | | | antibodies to GH. It is not | |
| | | | indicated to treat Secondary | |
| | | | IGFD resulting from GH | |
| | | | deficiency, malnutrition, hypoth | |
| Menotropins | Repronex ™ | Fertility Agents | For treatment of female infertility | 78617 |
| Methotrexate | | Immunomodulatory | Uveitis, DME | |
| mTOR inhibitors | | | | |
| Muromonab | Orthoclone OKT3 ™ | Immunomodulatory | For treatment of organ transplant | 23148 |
| | (Ortho Biotech) | Agents; | recipients, prevention of organ | |
| | | Immunosuppressive | rejection | |
| | | Agents | | |
| Natalizumab | Tysabri ™ | Immunomodulatory Agents | For treatment of multiple | 115334 |
| | | | sclerosis. | |
| Nepafenac | | Cyclooxygenase Inhibitors | | |
| Nesiritide | Natrecor ™ | Cardiac drugs | For the intravenous treatment of | 118921 |
| | | | patients with acutely | |
| | | | decompensated congestive | |
| | | | heart failure who have dyspnea | |
| | | | at rest or with minimal activity. | |
| Nilotinib | | Tyrosine Kinase Inhibitors | | 530 |
| NS398 | | Cyclooxygenase Inhibitors | | |
| Octreotide | Atrigel ™; | Anabolic Agents; | For treatment of acromegaly and | 42687 |
| | Longastatin ™; | Antineoplastic Agents, | reduction of side effects from | |
| | Sandostatin ™; | Hormonal; Gastrointestinal | cancer chemotherapy | |
| | Sandostatin LAR ™; | Agents; Hormone | | |
| | Sandostatin LAR ™ | Replacement Agents | | |
| | (Novartis) | | | |
| Omalizumab | Xolair ™ (Genentech | Anti-Asthmatic Agents; | For treatment of asthma caused | 29596 |
| | Inc) | Immunomodulatory Agents | by allergies | |
| Oprelvekin | Neumega ™; | Coagulants; Thrombotics | Increases reduced platelet levels | 45223 |
| | Neumega ™ (Genetics | | due to chemotherapy | |
| | Institute Inc) | | | |
| OspA lipoprotein | LYMErix ™ (SmithKline | Vaccines | For prophylactic treatment of | 95348 |
| | Beecham) | | Lyme Disease | |
| OT-551 | (Othera) | Anti-oxidant eyedrop | AMD | |
| Oxytocin | Oxytocin ™ (BAM | Anti-tocolytic Agents; | To assist in labor, elective labor | 12722 |
| | Biotech); Pitocin ™ | Labor Induction Agents; | induction, uterine contraction | |
| | (Parke-Davis); | Oxytocics | induction | |
| | Syntocinon ™ (Sandoz) | | | |
| Palifermin | Kepivance ™ (Amgen | Antimucositis Agents | For treatment of mucositis | 138885 |
| | Inc) | | (mouth sores) | |
| Palivizumab | Synagis ™ | Antiviral Agents | For treatment of respiratory | 63689 |
| | | | diseases casued by respiratory | |
| | | | syncytial virus | |
| Panitumumab | Vectibix ™; Vectibix ™ | Antineoplastic Agents | For the treatment of EGFR- | 134279 |
| | (Amgen) | | expressing, metastatic colorectal | |
| | | | carcinoma with disease | |
| | | | progression on or following | |
| | | | fluoropyrimidine-, oxaliplatin-, | |
| | | | and irinotecan- containing | |
| | | | chemotherapy regimens. | |
| Pazopanib | Votrient ™ (GSK) | Kinase inhibitor | Ocular neovascularization; | 437.517 |
| | | | ocular pain; keratoconjunctivis; | |
| | | | subfoveal choroidal | |
| | | | neovascularization secondary to | |
| | | | AMD | |
| PDGF inhibitor | (Jerini Ophthalmic); | Inhibitors of PDGF | AMD | |
| | (Ophthotech) | | | |
| PEDF (pigment | | | | |
| Ppithelium derived factor) | | | | |
| Pegademase bovine | Adagen ™ (Enzon Inc.) | Enzyme Replacement | For treatment of adenosine | 36512 |
| | | Agents | deaminase deficiency | |
| Pegaptanib | Macugen ™ | Oligonucleotide | For the treatment of neovascular | 103121 |
| | | | (wet) age-related macular | |
| | | | degeneration. | |
| Pegaspargase | Oncaspar ™ (Enzon | Antineoplastic Agents | For treatment of acute | 132.118 |
| | Inc) | | lymphoblastic leukemia | |
| Pegfilgrastim | Neulasta ™ (Amgen | Anti-Infective Agents; | Increases leukocyte production, | 28518 |
| | Inc.) | Antineutropenic Agents; | for treatment in non-myeloid | |
| | | Immunomodulatory Agents | cancer, neutropenia and bone | |
| | | | marrow transplant | |
| Peginterferon alfa-2a | Pegasys ™ (Hoffman- | Antineoplastic Agents; | For treatment of hairy cell | 57759 |
| | La Roche Inc) | Antiviral Agents; | leukemia, malignant melanoma, | |
| | | Immunomodulatory Agents | and AIDS-related Kaposi's | |
| | | | sarcoma. | |
| Peginterferon alfa-2b | PEG-Intron (Schering | Antineoplastic Agents; | For the treatment of chronic | 57759 |
| | Corp); Unitron PEG ™ | Antiviral Agents; | hepatitis C in patients not | |
| | | Immunomodulatory Agents | previously treated with interferon | |
| | | | alpha who have compensated | |
| | | | liver disease and are at least 18 | |
| | | | years of age. | |
| Pegvisomant | Somavert ™ (Pfizer Inc) | Anabolic Agents; Hormone | For treatment of acromegaly | 71500 |
| | | Replacement Agents | | |
| Pentoxifylline | | | | |
| Perindozril | | ACE Inhibitors | | |
| Pimecrolimus | | Limus Immunophilin | | |
| | | Binding Compounds | | |
| PKC (protein kinase C) | | | | |
| inhibitors | | | | |
| POT-4 | Potentia/Alcon | Complement Cascade | AMD | |
| | | Inhibitor (Factor C3) | | |
| Pramlintide | Symlin ™; Symlin ™ | | For the mealtime treatment of | 16988 |
| | (Amylin | | Type I and Type II diabetes in | |
| | Pharmaceuticals) | | combination with standard | |
| | | | insulin therapy, in patients who | |
| | | | have failed to achieve adequate | |
| | | | glucose control on insulin | |
| | | | monotherapy. | |
| Proteosome inhibitors | Velcade ™ | | Proteosome inhibitors | |
| Pyrrolidine | | | | |
| Quinopril | | ACE Inhibitors | | |
| Ranibizumab | Lucentis ™ | | For the treatment of patients with | 27043 |
| | | | neovascular (wet) age-related | |
| | | | macular degeneration. | |
| Rapamycin (siroliums) | (MacuSight) | Limus Immunophilin | AMD | |
| | | Binding Compounds | | |
| Rasburicase | Elitek ™; Elitek ™ | Antihyperuricemic Agents | For treatment of hyperuricemia, | 168.11 |
| | (Sanofi-Synthelabo | | reduces elevated plasma uric | |
| | Inc); Fasturtec ™ | | acid levels (from chemotherapy) | |
| Reteplase | Retavase ™ (Centocor); | Thrombolytic Agents | For lysis of acute pulmonary | 54732 |
| | Retavase ™ (Roche) | | emboli, intracoronary emboli and | |
| | | | management of myocardial | |
| | | | infarction | |
| Retinal stimulant | Neurosolve ™ | Retinal stimulants | AMD | |
| | (Vitreoretinal | | | |
| | Technologies) | | | |
| Retinoid(s) | | | | |
| Rituximab | MabThera ™; Rituxan ™ | Antineoplastic Agents | For treatment of B-cell non- | 33078 |
| | | | Hodgkins lymphoma (CD20 | |
| | | | positive) | |
| RNAI (RNA interference | | | | |
| of angiogenic factors) | | | | |
| Rofecoxib | Vioxx ™; Ceoxx ™; | Cyclooxygenase Inhibitors | | |
| | Ceeoxx ™ (Merck & | | | |
| | Co.) | | | |
| Rosiglitazone | | Thiazolidinediones | | |
| Ruboxistaurin | Eli Lilly | Protein Kinase C (PKC)-b | DME, diabetic peripheral | 469 |
| | | Inhibitor | retinopathy | |
| Salmon Calcitonin | Calcimar ™; Miacalcin ™ | Antihypocalcemic Agents; | For the treatment of post- | 57304 |
| | (Novartis) | Antiosteporotic Agents; | menopausal osteoporosis | |
| | | Bone Density | | |
| | | Conservation Agents | | |
| Sargramostim | Immunex ™; | Anti-Infective Agents; | For the treatment of cancer and | 46207 |
| | Leucomax ™ (Novartis); | Antineoplastic Agents; | bone marrow transplant | |
| | Leukine ™; Leukine ™ | Immunomodulatory Agents | | |
| | (Berlex Laboratories | | | |
| | Inc) | | | |
| SAR 1118 | SARCode | Immunomodulatory Agent | Dry eye, DME, conjunctivitis | |
| SDZ-RAD | | Limus Immunophilin | | |
| | | Binding Compounds | | |
| Secretin | SecreFlo ™; | Diagnostic Agents | For diagnosis of pancreatic | 50207 |
| | Secremax ™, | | exocrine dysfunction and | |
| | SecreFlo ™ (Repligen | | gastrinoma | |
| | Corp) | | | |
| Selective inhibitor of the | | | | |
| factor 3 complement | | | | |
| cascade | | | | |
| Selective inhibitor of the | | | | |
| factor 5 complement | | | | |
| cascade | | | | |
| Semaxanib | | Tyrosine Kinase Inhibitors | | 238 |
| Sermorelin | Geref ™ (Serono | Anabolic Agents; Hormone | For the treatment of dwarfism, | 47402 |
| | Pharma) | Replacement Agents | prevention of HIV-induced | |
| | | | weight loss | |
| Serum albumin iodinated | Megatope ™ (IsoTex | Imaging Agents | For determination of total blood | 39000 |
| | Diagnostics) | | and plasma volumes | |
| SF1126 | Semafore | PI3k/mTOR Inhibition | AMD, DME | |
| Sirolimus reformulation | (MacuSight) | Limus Immunophilin | AMD | |
| (rapamycin) | | Binding Compounds | | |
| siRNA molecule | (Quark | siRNA molecule synthetic | AMD | |
| synthetic, FTP-801i-14 | Pharmaceuticals) | | | |
| Somatropin recombinant | BioTropin ™ (Biotech | Anabolic Agents; Hormone | For treatment of dwarfism, | 71500 |
| | General); Genotropin ™ | Replacement Agents | acromegaly and prevention of | |
| | (Pfizer); Humatrope ™ | | HIV-induced weight loss | |
| | (Eli Lilly); Norditropin ™ | | | |
| | (Novo Nordisk); | | | |
| | Nutropin ™ (Genentech | | | |
| | Inc.); NutropinAQ ™ | | | |
| | (Genentech Inc.); | | | |
| | Protropin ™ (Genentech | | | |
| | Inc.); Saizen ™ (Serono | | | |
| | SA); Serostim ™; | | | |
| | Serostim ™ (Serono | | | |
| | SA); Tev-Tropin ™ | | | |
| | (GATE) | | | |
| Squalamine | | | | |
| Streptokinase | Streptase ™ (Aventis | Thrombolytic Agents | For the treatment of acute | 90569 |
| | Behringer GmbH) | | evolving transmural myocardial | |
| | | | infarction, pulmonary embolism, | |
| | | | deep vein thrombosis, arterial | |
| | | | thrombosis or embolism and | |
| | | | occlusion of arteriovenous | |
| | | | cannulae | |
| Sunitinib | | Tyrosine Kinase Inhibitors | | 398 |
| TA106 | Taligen | Complement Cascade | AMD | |
| | | Inhibitor (Factor B) | | |
| Tacrolimus | | Limus Immunophilin | | |
| | | Binding Compounds | | |
| Tenecteplase | TNKase ™ (Genentech | Thrombolytic Agents | For treatment of myocardial | 54732 |
| | Inc) | | infarction and lysis of | |
| | | | intracoronary emboli | |
| Teriparatide | Apthela ™; Forsteo ™; | Bone Density | For the treatment of | 66361 |
| | Forteo ™; Fortessa ™; | Conservation Agents | osteoporosis in men and | |
| | Opthia ™; Optia ™; | | postmenopausal women who | |
| | Optiah ™; Zalectra ™; | | are at high risk for having a | |
| | Zelletra ™ | | fracture. Also used to increase | |
| | | | bone mass in men with primary | |
| | | | or hypogonadal osteoporosis | |
| | | | who are at high risk for fracture. | |
| Tetrathiomolybdate | | | | |
| Thalidomide | Celgene | Anti-inflammatory, Anti- | Uveitis | |
| | | proliferative | | |
| [symbol] hyrotropin Alfa | Thyrogen ™ (Genzyme | Diagnostic Agents | For detection of residual or | 86831 |
| | Inc) | | recurrent thyroid cancer | |
| Tie-1 and Tie-2 kinase | | | | |
| inhibitors | | | | |
| Toceranib | | Tyrosine Kinase Inhibitors | | 396 |
| Tositumomab | Bexxar ™ (Corixa Corp) | Antineoplastic Agents | For treatment of non-Hodgkin's | 33078 |
| | | | lymphoma (CD20 positive, | |
| | | | follicular) | |
| TPN 470 analogue | | | | |
| Trastuzumab | Herceptin ™ | Antineoplastic Agents | For treatment of HER2-positive | 137912 |
| | (Genentech) | | pulmonary breast cancer | |
| Triamcinolone acetonide | Triesence ™ | Glucocorticoid | DME, For treatment of | 435 |
| | | | inflammation of the retina | |
| Troglitazone | | Thiazolidinediones | | |
| Tumistatin | | | | |
| Urofollitropin | Fertinex ™ (Serono | Fertility Agents | For treatment of female infertility | 78296 |
| | S.A.) | | | |
| Urokinase | Abbokinase ™; | Thrombolytic Agents | For the treatment of pulmonary | 90569 |
| | Abbokinase ™ (Abbott | | embolism, coronary artery | |
| | Laboratories) | | thrombosis and IV catheter | |
| | | | clearance | |
| Vandetanib | | Tyrosine Kinase Inhibitors | | 475 |
| Vasopressin | Pitressin ™; Pressyn ™ | Antidiuretics; Oxytocics; | For the treatment of enuresis, | 46800 |
| | | Vasoconstrictor Agents | polyuria, diabetes insipidus, | |
| | | | polydipsia and oesophageal | |
| | | | varices with bleeding | |
| Vatalanib | | Tyrosine Kinase Inhibitors | | 347 |
| VEGF receptor kinase | | | | |
| inhibitor | | | | |
| VEGF Trap | Aflibercept ™ | Genetically Engineered | DME, cancer, retinal vein | 96600 |
| | (Regneron | Antibodies | occlusion, choroidal | |
| | Pharmaceuticals, Bayer | | neovascularization, delay wound | |
| | Healthcare AG) | | healing, cancer treatment | |
| Visual Cycle Modulator | (Acucela) | Visual Cycle Modulator | AMD | |
| ACU-4229 | | | | |
| Vitamin(s) | | | | |
| Vitronectin receptor | | | | |
| antagonists | | | | |
| Volociximab | Ophthotech | alpha5beta1 Integrin | AMD | |
| | | Inhibitor | | |
| XL765 | Exelixis/Sanofi-Aventis | PI3k/mTOR Inhibition | AMD, DME | |

[symbol]

indicates data missing or illegible when filed

1. A device to treat an ocular condition of an eye, the device comprising:
a proximal region;
a tubular body coupled to the proximal region having an outer diameter configured to be inserted at least in part into the eye;
a reservoir in fluid communication with the tubular body and having a volume sized to receive an amount of a formulation of a therapeutic agent; and
one or more outlets in fluid communication with the reservoir and configured to release therapeutic amounts of the therapeutic agent into the eye for an extended time when the one or more outlets are positioned inside the eye.

2. The device of claim 1, wherein the formulation of the therapeutic agent is a free acid formulation.

3. The device of claim 2, wherein the free acid formulation is a solution configured to be injected into the reservoir volume after implantation.

4. The device of claim 2, wherein the free acid formulation is a free acid formulation of a prostaglandin analogue.

5. The device of claim 4, wherein the prostaglandin analogue is selected from the group consisting of travaprost, bimatoprost, tafluprost, and latanoprost.

6. The device of claim 4, wherein the solution is dissolved in concentrations higher than a solubility of a prodrug form of the prostaglandin analogue in water at pH 7.

7. The device of claim 2, wherein the free acid formulation has a higher solubility in aqueous formulation.

8. The device of claim 1, wherein the formulation of the therapeutic agent is a prostaglandin analogue comprising one or more solubilizing agents.

9. The device of claim 8, wherein the one or more solubilizing agents are selected from a group consisting of cyclodextrin, PEG, and ethanol.

10. The device of claim 1, wherein the outer diameter of the tubular body is configured to be inserted in the eye through a small gauge device.

11. The device of claim 1, wherein the outer diameter of the tubular body is configured to be inserted through an incision or opening in the eye that is no greater than about 0.5 mm.

12. The device of claim 1, wherein the volume of the reservoir is less than 5 ul.

13. The device of claim 1, wherein the extended time is at least 3 months.

14. The device of claim 2, wherein the free acid formulation is a suspension.

15. The device of claim 2, wherein the free acid formulation is a solid biodegradable pellet.

16. The device of claim 15, wherein the one or more outlets comprises a release control element.

17. The device of claim 16, further comprising a boundary layer of fluid between the solid biodegradable pellet and the release control element.

18. The device of claim 1, wherein the tubular body is a 5 mm long polyimide tube having a wall thickness of 0.127 mm and an outer diameter of 0.53 mm.

19. The device of claim 18, wherein the one or more outlets comprises a release control element or a porous membrane.

20. The device of claim 1, wherein the reservoir forms an interior of the tubular body.

21.-95. (canceled)
